(19) United States
(12) Patent Application Publication
(10) Pub. No.: US 2026/0166095 A1
(43) Pub. Date: Jun. 18, 2026

(54) COMPOSITIONS FOR TREATMENT OF GLIOMA AND USES THEREOF

(71) Applicant: Pacific Marine Biotech LLC, Torrington, CT (US)

(72) Inventor: Samuel Joseph GRANT, Port Vila (VU)

(21) Appl. No.: 19/531,468

(22) Filed: Feb. 5, 2026

Related U.S. Application Data
(63) Continuation of application No. PCT/US25/40083, filed on Jul. 31, 2025.
(60) Provisional application No. 63/678,697, filed on Aug. 2, 2024.
(60) Provisional application No. 63/761,698, filed on Feb. 21, 2025.

(51) Int. Cl. A61K 35/616; A61K 31/175; A61K 31/495; A61K 35/655; A61K 36/03; A61N 5/10; A61P 35/00; C07K 16/22

(52) U.S. Cl. CPC A61K 35/616; A61K 31/175; A61K 31/495; A61K 35/655; A61K 36/03; A61N 5/10; A61P 35/00; C07K 16/22

(57) ABSTRACT

The present disclosure provides for compositions comprising a combination of marine biomass extracts for use in the treatment of brain cancer. In some instances, the brain cancer is glioma. In some instances, the glioma is glioblastoma. Further provided herein are biomass compositions having extracts from Holothuria scabra, Holothuria nobilis, Heliocidaris erythrogramma, Styela clava, and Sargassum pallidum. Moreover, in such compositions the majority components may be extracts from sea cucumber.

### CROSS REFERENCE TO RELATED APPLICATIONS

This application claims the benefit of priority and is a continuation of International Application No. PCT/US2025/040083, filed Jul. 31, 2025, which claims the benefit of priority to Provisional Application Ser. No. 63/678,697 filed on Aug. 2, 2024, and Provisional Application Ser. No. 63/761,698 filed on Feb. 21, 2025, the disclosures of which are incorporated herein by reference in their entirety.

### BACKGROUND

A brain cancer is a growth of cells in the brain or near it. Gliomas are a form of brain cancer. A glioma is a tumor that forms when glial cells grow out of control. Normally, these cells support nerves and help your central nervous system work. Gliomas usually grow in the brain, but can also form in the spinal cord. Gliomas are malignant (cancerous), but some can be very slow growing. Glioblastoma is a type of glioma originating in astrocytes and is classified as high-grade. Glioblastoma (GBM) is a complex, deadly, and treatment-resistant neoplasm. The median overall survival is 14.6 months, with progression common within the first two years of diagnosis. The current standard of care consists of maximal safe resection or biopsy, followed by concomitant radiation therapy with temozolomide (TMZ), followed by maintenance TMZ for 6 to 12 months with or without the addition of tumor treating fields. Prognosis is improved in subjects who undergo gross total resection and whose tumors demonstrate 06-methylguanine-DNA methyltransferase (MGMT) promoter methylation and isocitrate dehydrogenase (IDH) mutations. In those subjects with MGMT promotor hypomethylation and lacking IDH mutations, the outcomes are notably worse. Standard therapies for recurrent disease remain limited. Thus, there is a need for improved therapies for gliomas, including glioblastoma.

### BRIEF SUMMARY

Provided herein are methods of treatment or reduction of a brain cancer in a subject in need thereof, the method comprising administering to a subject a therapeutically effective dose of a biomass composition comprising a biomass material, wherein the biomass material comprises: biomass material from a plurality of marine invertebrates, wherein the plurality of marine invertebrates comprises at least one sea cucumber, and wherein at least 80% dry weight biomass material is from the at least on sea cucumber; and algae biomass material. Further provided herein are methods, wherein the at least one sea cucumber comprises Holothuria scabra or Holothuria nobilis. Further provided herein are methods, wherein the plurality of marine invertebrates comprises further comprises a sea urchin and/or sea squirt. Further provided herein are methods, wherein the sea urchin comprises Heliocidaris erythrogramma. Further provided herein are methods, wherein the sea squirt comprises Styela clava. Further provided herein are methods, where the algae biomass material comprises Sargassum pallidum. Further provided herein are methods, wherein the biomass material is derived from Holothuria scabra, Holothuria nobilis, Heliocidaris erythrogramma, Styela clava, and Sargassum pallidum to the subject. Further provided herein are methods, wherein the brain cancer is a glioma. Further provided herein are methods, wherein the glioma comprises a glioblastoma, an astrocytoma, or a diffuse intrinsic pontine glioma. Further provided herein are methods, wherein the glioblastoma comprises a glioblastoma multiforme (GBM), a newly diagnosed glioblastoma, a giant cell glioblastoma, a gliosarcoma, a drug-resistant glioblastoma, a recurrent glioblastoma, a relapsed glioblastoma, or a refractory glioblastoma. Further provided herein are methods, wherein the astrocytoma comprises a grade I astrocytoma, a grade II astrocytoma, a grade III astrocytoma, or a grade IV astrocytoma. Further provided herein are methods, wherein the astrocytoma is a grade IV astrocytoma. Further provided herein are methods, wherein the grade IV astrocytoma does not comprise an isocitrate dehydrogenase 1 (IDH1) mutation. Further provided herein are methods, wherein the grade IV astrocytoma comprises an isocitrate dehydrogenase 1 (IDH1) mutation. Further provided herein are methods, wherein the brain cancer is an oligodendroglioma, an ependymoma, a medulloblastoma, a choroid plexus carcinoma, a pineoblastoma, a menmigioma, an acoustic neuroma, or a craniopharyngioma. Further provided herein are methods, wherein the biomass composition is lyophilized. Further provided herein are methods, wherein the biomass composition is a powder. Further provided herein are methods, wherein the biomass composition is administered orally. Further provided herein are methods, wherein the biomass composition is administered intravenously. Further provided herein are methods, wherein the administering of the therapeutically effective dose of the biomass composition comprises oral administration. Further provided herein are methods, wherein the administering is daily or twice daily. Further provided herein are methods, wherein the biomass composition is administered to the subject in one or two oral doses per day. Further provided herein are methods, wherein the biomass composition is administered to the subject in one or two intravenous doses per day. Further provided herein are methods, further comprising administering to the subject an additional cancer therapy. Further provided herein are methods, wherein the additional cancer therapy is administered prior to before, simultaneously with, or after the administering of the biomass composition. Further provided herein are methods, wherein the additional cancer therapy comprises a chemotherapy or a radiation therapy. Further provided herein are methods, wherein the additional cancer therapy comprises a chemotherapy and a radiation therapy. Further provided herein are methods, wherein the chemotherapy comprises temozolomide, carmustine, bevacizumab, or lomustine. Further provided herein are methods, wherein the additional therapy is temozolomide.

Further provided herein are methods, wherein the additional therapy is lomustine. Further provided herein are methods, wherein the radiation therapy comprises tumor treating fields (TTF), Intensity-modulated radiation therapy (IMRT), and Image-guided radiation therapy (IGRT). Further provided herein are methods, wherein a majority of biomass material is from the at least one sea cucumber, wherein the at least one sea cucumber comprises two species of sea cucumbers. Further provided herein are methods, wherein the biomass material comprises an extract derived from at least two species of sea cucumber, a species of seagrass, a species of sea squirt, and a species of sea urchin. Further provided herein are methods, wherein the extract derived from the at least two species of sea cucumber comprises an extract from Holothuria scabra and an extract from Holothuria nobilis. Further provided herein are methods, wherein the biomass composition comprises from about 20% w/w to about 40% w/w of the extract from Holothuria scabra. Further provided herein are methods, wherein the extract from Holothuria scabra is 40% w/w of the biomass composition. Further provided herein are methods, wherein the biomass composition comprises from about 25% w/w to about 50% w/w of the extract from Holothuria nobilis. Further provided herein are methods, wherein the extract from Holothuria nobilis is 45% w/w of the biomass composition. Further provided herein are methods, wherein the extract derived from the species of seagrass comprises an extract from Sargassum pallidum. Further provided herein are methods, wherein the biomass composition comprises from about 1% w/w to about 10% w/w of the extract from Sargassum pallidum. Further provided herein are methods, wherein the extract from Sargassum pallidum is 5% w/w of the biomass composition. Further provided herein are methods, wherein the extract derived from the species of sea squirt comprises an extract from Styela clava. Further provided herein are methods, wherein the biomass composition comprises from about 1% w/w to about 10% w/w of the extract from Styela clava. Further provided herein are methods, wherein the extract from Styela clava is 5% w/w of the biomass composition. Further provided herein are methods, wherein the extract derived from the species of sea urchin comprises an extract from Heliocidaris erythrogramma. Further provided herein are methods, wherein the biomass composition comprises from about 1% w/w to about 10% w/w of the extract from Heliocidaris erythrogramma. Further provided herein are methods, wherein the extract from Heliocidaris erythrogramma is 5% w/w of the biomass composition. Further provided herein are methods, wherein the extract derived from the at least two species of sea cucumber, the species of seagrass, the species of sea squirt, and the species of sea urchin comprises a whole-body extract. Further provided herein are methods, wherein the biomass composition is unfiltered prior to the administering to the subject. Further provided herein are methods, wherein the biomass composition is filtered prior to the administering to the subject. Further provided herein are methods, wherein the biomass composition is not centrifuged prior to the administering to the subject. Further provided herein are methods, wherein the biomass composition is centrifuged prior to the administering to the subject. Further provided herein are methods, wherein the biomass composition further comprises a solubilizing agent. Further provided herein are methods, wherein the solubilizing agent is dimethyl sulfoxide (DMSO).

Provided herein are methods of treatment or reduction of a brain cancer in a subject in need thereof, the method comprising administering to a subject: a biomass composition comprising a biomass material derived from Holothuria scabra, Holothuria nobilis, Heliocidaris erythrogramma, Styela clava, and Sargassum pallidum; and temozolomide, or a salt thereof. Further provided herein are methods, wherein the biomass composition is administered prior to before, simultaneously with, or after the administering temozolomide, or the salt thereof. Further provided herein are methods, wherein the biomass composition is administered orally. Further provided herein are methods, wherein the biomass composition is administered intravenously.

Provided herein are methods of treatment or reduction of a brain cancer in a subject in need thereof, the method comprising administering to a subject: a biomass composition comprising a biomass material derived from Holothuria scabra, Holothuria nobilis, Heliocidaris erythrogramma, Styela clava, and Sargassum pallidum to the subject; and lomustine, or a salt thereof. Further provided herein are methods, wherein the biomass composition is administered prior to before, simultaneously with, or after the administering lomustine, or the salt thereof. Further provided herein are methods, wherein the biomass composition is administered orally. Further provided herein are methods, wherein the biomass composition is administered intravenously.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a cell viability assay for a human glioma cell line (U87). The X-axis represents the concentration of the biomass composition, and the Y-axis represents the cell viability as a percentage of untreated cells.

FIGS. 2A-2C depicts flow cytometry data of propidium iodide (PI) stained U87 cells treated for 24 hours with the biomass compositions disclosed herein. FIG. 2A represents cells untreated cells; FIG. 2B represents cells treated with 100 mg/ml of the biomass composition; FIG. 2C represents cells treated with 200 mg/ml of the biomass composition. The X-axis represents fluorescence pulse heights (FL2-H), and the Y-axis represents cell count.

FIG. 3 depicts a series of serial contrast enhanced T1-weighted Magnetic Resonance Imaging (MRI) images from a subject before a during co-administration of the biomass composition according to the methods disclosed herein and chemotherapy plus radiation therapy (ChemoRT).

FIGS. 4A-4C shows a subject's glioblastoma before (FIGS. 4A-4B) and after administration of the formulations described herein (FIG. 4C) according to the methods disclosed herein. FIG. 4A depicts a series of serial contrast enhanced T1-weighted MRI images a subject using only the standard of care treatment, and said subject after administration of the biomass composition disclosed herein. FIG. 4B is a back view MRI image showing the subject's glioblastoma before administration of the biomass composition disclosed herein. FIG. 4C is a back view MRI image of the subject's glioblastoma after administration of the biomass composition disclosed herein.

FIGS. 5A-5B shows a subject's diffuse intrinsic pontine glioma before (FIG. 5A) and after administration of the formulations described herein (FIG. 5B) according to the methods disclosed herein.

FIG. 6 depicts a histogram of disease progression. Each bar on the X-axis represents individual subjects who underwent treatment as disclosed herein, and Y-axis represents the number of months.

FIG. 7 depicts a histogram mean overall survival. Each bar on the X-axis represents individual subjects who underwent treatment as disclosed herein, and Y-axis represents the number of months.

FIG. 8 depicts a histogram of overall survival. Each bar on the X-axis represents individual subjects who underwent treatment as disclosed herein, and Y-axis represents the number of months.

FIGS. 9A-9D depicts the results of the in vitro anti-tumor activity of the filtered and unfiltered biomass compositions disclosed herein on two representative glioblastoma cell lines. FIG. 9A depicts the dose-dependent fold change of cell growth in LN229 cells after treatment with filtered biomass composition. The X-axis depicts the % v/v concentration of the biomass composition, and Y-axis depicts the fold change of cell growth. FIG. 9B depicts the dose-dependent fold change of cell growth in LN229 cells after treatment with unfiltered biomass composition. The X-axis depicts the % v/v concentration of the biomass composition, and Y-axis depicts the fold change of cell growth. FIG. 9C depicts the dose-dependent fold change of cell growth in GBM8 cells after treatment with filtered biomass composition. The X-axis depicts the % v/v concentration of the biomass composition, and Y-axis depicts the fold change of cell growth. FIG. 9D depicts the dose-dependent fold change of cell growth in GBM8 cells after treatment with unfiltered biomass composition. The X-axis depicts the % v/v concentration of the biomass composition, and Y-axis depicts the fold change of cell growth.

FIG. 10 depicts a graph showing off-target toxicity of the biomass compositions on an organotypic brain slice culture. The X-axis depicts the dose (%) of the biomass composition, and Y-axis depicts the survival (%).

FIGS. 11A-11B depicts the results of the in vitro anti-tumor activity of the 0.45 uM filtered biomass composition as compared to unfiltered biomass composition on a representative glioblastoma cell line (LN229). FIG. 11A depicts the dose-dependent fold change of cell growth in LN229 cells after treatment with filtered biomass composition. The X-axis depicts the % v/v concentration of the biomass composition, and Y-axis depicts the fold change of cell growth. FIG. 11B depicts the dose-dependent fold change of cell growth in LN229 cells after treatment with unfiltered biomass composition. The X-axis depicts the % v/v concentration of the biomass composition, and Y-axis depicts the fold change of cell growth.

FIGS. 12A-12B depicts the results of the in vitro anti-tumor activity of the 0.45 uM filtered biomass composition as compared to unfiltered biomass composition on a representative glioblastoma cell line (U87). FIG. 12A depicts the dose-dependent fold change of cell growth in U87 cells after treatment with filtered biomass composition. The X-axis depicts the % v/v concentration of the biomass composition, and Y-axis depicts the fold change of cell growth. FIG. 12B depicts the dose-dependent fold change of cell growth in U87 cells after treatment with unfiltered biomass composition. The X-axis depicts the % v/v concentration of the biomass composition, and Y-axis depicts the fold change of cell growth.

FIG. 13 depicts a graph showing off-target toxicity of the 0.45 uM filtered biomass composition as compared to unfiltered biomass composition on an organotypic brain slice culture. The X-axis depicts the dose (%) of the biomass composition, and Y-axis depicts the survival (%).

FIGS. 14A-14B depicts the results of the in vitro anti-tumor activity of the 0.22 uM filtered biomass composition as compared to unfiltered biomass composition on a representative glioblastoma cell line (GBM8). FIG. 14A depicts the dose-dependent fold change of cell growth in GBM8 cells after treatment with filtered biomass composition. The X-axis depicts the % v/v concentration of the biomass composition, and Y-axis depicts the fold change of cell growth. FIG. 14B depicts the dose-dependent fold change of cell growth in GBM8 cells after treatment with unfiltered biomass composition. The X-axis depicts the % v/v concentration of the biomass composition, and Y-axis depicts the fold change of cell growth.

FIGS. 15A-15D depicts the results of the in vitro anti-tumor activity of different concentrations of filtered and unfiltered biomass compositions disclosed herein on U87 cells alone, or U87 cells grafted on an organotypic brain slice culture (OBSC) platform. FIG. 15A depicts the dose-dependent tumor kill (%) of U87 cells after treatment with filtered biomass composition. The X-axis depicts the % v/v concentration of the biomass composition, and Y-axis depicts the tumor kill (%). FIG. 15B depicts the dose-dependent tumor kill (%) of U87 cells after treatment with unfiltered biomass composition. The X-axis depicts the % v/v concentration of the biomass composition, and Y-axis depicts the tumor kill (%). FIG. 15C depicts the dose-dependent tumor kill (%) of U87 cells grafted on OBSC platform after treatment with filtered biomass composition. The X-axis depicts the % v/v concentration of the biomass composition, and Y-axis depicts the tumor kill (%). FIG. 15D depicts the dose-dependent tumor kill (%) of U87 cells grafted on OBSC platform after treatment with unfiltered biomass composition. The X-axis depicts the % v/v concentration of the biomass composition, and Y-axis depicts the tumor kill (%).

FIGS. 16A-16D depicts the results of the in vitro anti-tumor activity of the 0.45 uM filtered biomass composition as compared to unfiltered biomass composition on two representative glioblastoma cell lines. FIG. 16A depicts the dose-dependent fold change of cell growth in Kr158b cells after treatment with 0.45 uM filtered biomass composition. The X-axis depicts the % v/v concentration of the biomass composition, and Y-axis depicts the fold change of cell growth. FIG. 16B depicts the dose-dependent fold change of cell growth in Kr158b cells after treatment with unfiltered biomass composition. The X-axis depicts the % v/v concentration of the biomass composition, and Y-axis depicts the fold change of cell growth. FIG. 16C depicts the dose-dependent fold change of cell growth in GL261 cells after treatment with 0.45 uM filtered biomass composition. The X-axis depicts the % v/v concentration of the biomass composition, and Y-axis depicts the fold change of cell growth. FIG. 16D depicts the dose-dependent fold change of cell growth in GL261 cells after treatment with unfiltered biomass composition. The X-axis depicts the % v/v concentration of the biomass composition, and Y-axis depicts the fold change of cell growth.

FIGS. 17A-17B depicts the effects of the centrifugation on anti-tumor activity of the biomass compositions described herein. FIG. 17A depicts the effects of the eight centrifugation speeds on anti-tumor activity of the biomass compositions on LN229 cells as compared to a no centrifuge control and a 2-hour settled (no centrifuge) control. The X-axis depicts concentration (% v/v), and for each condition increasing concentration is depicted by the triangle. The Y-axis depicts the fold change of cell growth. FIG. 17B depicts the effects of the eight centrifugation speeds on anti-tumor activity of the biomass compositions on GL261 cells as compared to a no centrifuge control and a 2-hour settled (no centrifuge) control. The X-axis depicts concentration (% v/v), and for each condition increasing concentration is depicted by the triangle. The Y-axis depicts the fold change of cell growth.

FIG. 18 depicts the results of the in vitro anti-tumor activity of the unfiltered and centrifuged biomass compositions as compared to the unfiltered and non-centrifuged biomass composition. The X-axis depicts the % v/v concentration of the biomass composition, and Y-axis depicts the fold change of cell growth.

FIG. 19 depicts the results of the in vitro anti-tumor activity of the biomass composition mixed with a solubilizing agent (dimethyl sulfoxide (DMSO)), as compared to the biomass composition without the DMSO added. The X-axis depicts the % v/v concentration of the biomass composition with 0% DMSO, 3% DMSO, and 10% DMSO, and Y-axis depicts the fold change of cell growth.

FIGS. 20A-20F depicts results on in vivo toxicity studies after treatment with biomass compositions. FIG. 20A depicts weight changes in immunocompromised mice after daily treatment with a saline control. The X-axis depicts the measurement day, and the Y-axis depicts the mouse weight (grams). FIG. 20B depicts weight changes in immunocompromised mice after daily treatment with a human dose equivalent of biomass compositions described herein. The X-axis depicts the measurement day, and the Y-axis depicts the mouse weight (grams). FIG. 20C depicts weight changes in immunocompromised mice after daily treatment with a high dose equivalent of biomass compositions described herein. The X-axis depicts the measurement day, and the Y-axis depicts the mouse weight (grams). FIG. 20D depicts weight changes in immunocompetent mice after daily treatment with a saline control. The X-axis depicts the measurement day, and the Y-axis depicts the mouse weight (grams). FIG. 20E depicts weight changes in immunocompetent mice after daily treatment with a human dose equivalent of biomass compositions described herein. The X-axis depicts the measurement day, and the Y-axis depicts the mouse weight (grams). FIG. 20F depicts weight changes in immunocompetent mice after daily treatment with a high dose equivalent of biomass compositions described herein. The X-axis depicts the measurement day, and the Y-axis depicts the mouse weight (grams).

FIGS. 21A-21B depict the mean values of body weights changes for both mouse strains over 28 days of treatment with a low dose or high dose of the biomass composition, as compared to a PBS control. FIG. 21A depicts the mean values of body weight changes for immunocompromised mice over a 28-day period. The X-axis depicts the number of days, and the Y-axis depicts the mouse weight in grams. At the −6 day time point, the bottom line represents the PBS control, the middle line represents the high dose, and the top line represents the low dose. FIG. 21B depicts the mean values of body weight changes for immunocompetent mice over a 28-day period. The X-axis depicts the number of days, and the Y-axis depicts the mouse weight in grams. At the −6 day time point, the bottom line represents the PBS control, the middle line represents the low dose, and the top line represents the high dose.

FIGS. 22A-22F depict the complete blood count (CBC) panel for both mouse strains at day 28, after treatment with a low dose or high dose of the biomass composition, as compared to a PBS control. FIGS. 22A-22B depicts the individual white blood cell counts for immunocompromised mice (FIG. 22A), and immunocompetent mice (FIG. 22B). The X-axis shows each white blood cell group, and the Y-axis shows the percent of cells. The black bar represents the PBS control, the light grey bar represents the low dose of the biomass composition, and the dark grey bar represents the high dose of the biomass composition. FIGS. 22C-22D depicts the liver function tests for immunocompromised mice (FIG. 22C), and immunocompetent mice (FIG. 22D). The X-axis shows each biomarker tested, and the Y-axis shows the units per liter (U/L). The black bar represents the PBS control, the light grey bar represents the low dose of the biomass composition, and the dark grey bar represents the high dose of the biomass composition.

FIGS. 22E-22F depicts the liver function tests for immunocompromised mice (FIG. 22E), and immunocompetent mice (FIG. 22F). The X-axis shows each biomarker tested, and the Y-axis shows the milligrams (mg) per deciliter (dL) (mg/dL). The black bar represents the PBS control, the light grey bar represents the low dose of the biomass composition, and the dark grey bar represents the high dose of the biomass composition.

FIGS. 23A-23E depict dose-dependent decrease in percent survival of five different types of brain cancer cell lines after treatment with the biomass compositions disclosed herein. FIG. 23A depicts the percent survival of LN229 cells after treatment with increasing doses of the biomass compositions disclosed herein. FIG. 23B depicts the percent survival of GL261 cells after treatment with increasing doses of the biomass compositions disclosed herein. FIG. 23C depicts the percent survival of U87 cells after treatment with increasing doses of the biomass compositions disclosed herein. FIG. 23D depicts the percent survival of KR158b cells after treatment with increasing doses of the biomass compositions disclosed herein. FIG. 23E depicts the percent survival of GBM8 cells after treatment with increasing doses of the biomass compositions disclosed herein. The X-axis shows concentration (v/v %), and the Y-axis shows survival (%).

FIGS. 24A-24D depict the in vivo efficacy of the biomass compositions disclosed herein in immunocompetent (C57B/L6) and immunocompromised (NSG) GL261 tumor bearing mice. FIG. 24A depicts the daily tumor growth for immunocompromised mice over a 15-day period. The X-axis shows days after treatment, and the Y-axis depicts total flux (p/s). FIG. 24B depicts the daily tumor growth for immunocompetent mice over a 15-day period. The X-axis shows days after treatment, and the Y-axis depicts total flux (p/s). FIG. 24C depicts the mean values of body weight changes for immunocompromised mice over a 15-day period. The X-axis shows days after treatment, and the Y-axis depicts the mouse weight in grams. FIG. 24D depicts the mean values of body weight changes for immunocompetent mice over a 15-day period. The X-axis shows days after treatment, and the Y-axis depicts the mouse weight in grams.

FIGS. 25A-25D depict the synergistic effects of the biomass compositions disclosed herein and an additional therapy on percent survival in LN229 cells. FIG. 25A depicts a table describing the percent survival of LN229 cells at increasing concentrations of the biomass composition and the additional therapy, temozolomide. Columns 2-7 shows the concentration of temozolomide (uM), and rows 2-7 shows the concentration of biomass composition (% v/v). FIG. 25B is a graph depicting the ZIP synergy score comparing the combined effects of the biomass composition and temozolomide. The X-axis shows the concentration of the biomass composition (uM), and the Y-axis shows the concentration of temozolomide (uM). FIG. 25C depicts a table describing the percent survival of LN229 cells at increasing concentrations of the biomass composition and the additional therapy, lomustine. Columns 2-7 shows the concentration of lomustine (uM), and rows 2-7 shows the concentration of biomass composition (% v/v). FIG. 25D is a graph depicting the ZIP synergy score comparing the combined effects of the biomass composition and lomustine. The X-axis shows the concentration of the biomass composition (uM), and the Y-axis shows the concentration of lomustine (uM).

### DETAILED DESCRIPTION

The present disclosure is generally directed to compositions and methods for treating gliomas using combination compositions comprising marine product extracts derived from one or more organisms. The marine product extracts may contain biomass material from animals and plants. In particular, provided herein are compositions and methods for treating brain cancers comprising administering to a subject in need thereof a biomass composition comprising a mixture of biomass material derived one or more organisms. In some embodiments, the one or more marine organisms comprise a marine invertebrate. In some embodiments, the one or more marine organisms comprise a plurality of marine invertebrates. In some embodiments, the plurality of marine invertebrates comprises Holothuria scabra, Holothuria nobilis, Heliocidaris erythrogramma, Styela clava, and Sargassum pallidum to the subject. In some embodiments, brain cancers comprise gliomas. Also provided herein are methods for ameliorating symptoms associated with gliomas comprising administering to a subject in need thereof a composition comprising a mixture of biomass material derived from Holothuria scabra, Holothuria nobilis, Heliocidaris erythrogramma, Styela clava, and Sargassum pallidum to the subject. The biomass composition as described herein is formulated in many dosage forms, such as liquid, gel, capsule, tablet, powder suppository, dispersions or suspensions. In some embodiments, the formulation comprises a suspension.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure may be described in the context of a single embodiment, the features can also be provided separately or in any suitable combination. Conversely, although the present disclosure may be described herein in the context of separate embodiments for clarity, the present disclosure can also be implemented in a single embodiment.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or apparent from context, as used herein, the term “about” in reference to a number or range of numbers is understood to mean the stated number and numbers +/−10% thereof, or 10% below the lower listed limit and 10% above the higher listed limit for the values listed for a range.

The term “subject” refers to an animal, including, but not limited to, a primate (e.g., human or non-human primate), cow, sheep, goat, horse, dog, cat, rabbit, rat, or mouse.

The terms “treat,” “treating,” and “treatment” is meant to include alleviating or abrogating a disorder, disease, or condition; or one or more of the symptoms associated with the disorder, disease, or condition; or alleviating or eradicating the cause(s) of the disorder, disease, or condition itself.

The term “therapeutically effective amount” refer to the amount of a compound that, when administered, is sufficient to prevent development of, or alleviate to some extent, one or more of the symptoms of the disorder, disease, or condition being treated.

### Compositions

Provided herein are biomass compositions comprising a mixture of biomass material derived from one or more marine organisms. In some embodiments, the one or more marine organisms comprise a marine invertebrate. In some embodiments, the one or more marine organisms comprise a plurality of marine invertebrates. In some embodiments, the one or more marine organisms comprises Holothuria scabra, Holothuria nobilis, Heliocidaris erythrogramma, Styela clava, and Sargassum pallidum. The biomass composition described herein comprise one or more marine-derived products. Marine-derived products contain compounds that have shown activity against neoplastic processes at the cellular and molecular level, and within the tumor microenvironment. However, many marine-derived products comprise toxins that have adverse side effects when accumulated in the body of a subject. For example, the adverse side effects include, but is not limited to, severe burning pain, localized edema, erythema, warmth, and bleeding. In severe cases, nausea, vomiting, paresthesia, muscular paralysis, respiratory distress, kidney disease, and organ failure. The biomass compositions described herein was formulated to reduce adverse side effects associate with accumulation of marine-derived products in the body of a subject. In some embodiments, the biomass compositions described herein provide a mixture of biomass material used for the treatment of a disease without observing the toxic side effect associated with one or more source(s) from the biomass material.

Also provided herein is a biomass composition comprising a mixture of biomass material derived from Holothuria scabra, Holothuria nobilis, Heliocidaris erythrogramma, Styela clava, and Sargassum pallidum. In some embodiments, the biomass composition comprises one or more purified compounds derived from Holothuria scabra. In some embodiments, the biomass composition comprises one or more purified compounds derived from Holothuria nobilis. In some embodiments, the biomass composition comprises one or more purified compounds derived from Heliocidaris erythrogramma. In some embodiments, the biomass composition comprises one or more purified compounds derived from Styela clava. In some embodiments, the biomass compositions disclosed herein is lyophilized. In some embodiments, the biomass composition comprises one or more purified compounds derived from Sargassum pallidum. In some embodiments, the one or more purified compounds comprise Cucumarioside A2-2, Fucoidan, Frondoside A, Echinoside A, Ecteinascidin 743, Holothurin A, Phlorotanins, Stichoposide C, Stomopneulactone D, Terminoside A, and Trabetectedin.

### Sea Cucumber

Provided herein the biomass composition comprises one or more sea cucumber derivatives from about 1% to about 99% as measured by weight and/or volume. In some embodiments, the biomass composition comprises one or more sea cucumber derivatives from about 1% to about 99% as measured by dry weight. In some embodiments, the biomass composition is about: 10%, 15%, 20%, 25%, 30%, 35%, 40%, 45%, 50%, 55%, 60%, 65%, 70%, 75%, 80%, 85%, 90%, 95% or 99% of sea cucumber extract. In some embodiments, the majority of the biomass composition comprises one or more sea cucumber derivatives. In some embodiments, the biomass composition comprises an extract from sea cucumber. In some embodiments, the extract from sea cucumber comprises an extract from the group consisting of: Holothuria scabra, Holothuria nobilis, Holothuria mexicana, Holothuria californica, Stichopus chlorontus, Stichopus horrens, Stichopus japonicus, Apostichopus japonicus, or Cucumaria japonica.

In some embodiments, the biomass composition comprises from 1% to 99% of a visceral extract, gonad extract, testes extract, ovary extract or combination of extracts of Holothuria scabra.

In some embodiments, the biomass composition comprises from 10% to 99% of a visceral extract, gonad extract, testes extract, ovary extract or combination of extracts of Holothuria scabra. In some embodiments, the biomass composition comprises from 20% to 99% of a visceral extract, gonad extract, testes extract, ovary extract or combination of extracts of Holothuria scabra. In some embodiments, the biomass composition comprises from 40% to 99% of a visceral extract, gonad extract, testes extract, ovary extract or combination of extracts of Holothuria scabra. In some embodiments, the biomass composition comprises from 20% to 80% of a visceral extract, gonad extract, testes extract, ovary extract or combination of extracts of Holothuria scabra. In some embodiments, the biomass composition comprises from 30% to 60% of a visceral extract, gonad extract, testes extract, ovary extract or combination of extracts of Holothuria scabra. In some embodiments, the biomass composition comprises from 40% to 50% of a visceral extract, gonad extract, testes extract, ovary extract or combination of extracts of Holothuria scabra. In some embodiments, the biomass composition comprises about 40% of a visceral extract, gonad extract, testes extract, ovary extract or combination of extracts of Holothuria scabra. In some embodiments, the biomass composition comprises an extract from a whole body of Holothuria scabra. In some embodiments, the biomass composition comprises about 40% of a visceral extract, gonad extract, testes extract, ovary extract or combination of extracts of Holothuria scabra. In some embodiments, the biomass composition comprises about 45% of a visceral extract, gonad extract, testes extract, ovary extract or combination of extracts of Holothuria scabra. In some embodiments, the biomass composition comprises an extract from the whole body of Holothuria scabra.

In some embodiments, the biomass composition comprises from 1% to 99% of a visceral extract, gonad extract, testes extract, ovary extract or combination of extracts of Holothuria nobilis.

In some embodiments, the biomass composition comprises from 10% to 99% of a visceral extract, gonad extract, testes extract, ovary extract or combination of extracts of Holothuria nobilis. In some embodiments, the biomass composition comprises from 20% to 99% of a visceral extract, gonad extract, testes extract, ovary extract or combination of extracts of Holothuria nobilis. In some embodiments, the biomass composition comprises from 40% to 99% of a visceral extract, gonad extract, testes extract, ovary extract or combination of extracts of Holothuria nobilis. In some embodiments, the biomass composition comprises from 20% to 80% of a visceral extract, gonad extract, testes extract, ovary extract or combination of extracts of Holothuria nobilis. In some embodiments, the biomass composition comprises from 30% to 60% of a visceral extract, gonad extract, testes extract, ovary extract or combination of extracts of Holothuria nobilis. In some embodiments, the biomass composition comprises from 40% to 50% of a visceral extract, gonad extract, testes extract, ovary extract or combination of extracts of Holothuria nobilis. In some embodiments, the biomass composition comprises about 40% of a visceral extract, gonad extract, testes extract, ovary extract or combination of extracts of Holothuria nobilis. In some embodiments, the biomass composition comprises about 45% of a visceral extract, gonad extract, testes extract, ovary extract or combination of extracts of Holothuria nobilis. In some embodiments, the biomass composition comprises an extract from the whole body of Holothuria nobilis.

In some embodiments, the biomass composition comprises from 1% to 99% of a visceral extract, gonad extract, testes extract, ovary extract or combination of extracts of Holothuria mexicana. In some embodiments, the biomass composition comprises from 10% to 99% of a visceral extract, gonad extract, testes extract, ovary extract or combination of extracts of Holothuria mexicana. In some embodiments, the biomass composition comprises from 20% to 99% of a visceral extract, gonad extract, testes extract, ovary extract or combination of extracts of Holothuria mexicana. In some embodiments, the biomass composition comprises from 40% to 99% of a visceral extract, gonad extract, testes extract, ovary extract or combination of extracts of Holothuria mexicana. In some embodiments, the biomass composition comprises from 60% to 99% of a visceral extract, gonad extract, testes extract, ovary extract or combination of extracts of Holothuria mexicana. In some embodiments, the biomass composition comprises from 20% to 80% of a visceral extract, gonad extract, testes extract, ovary extract or combination of extracts of Holothuria mexicana. In some embodiments, the biomass composition comprises from 30% to 60% of a visceral extract, gonad extract, testes extract, ovary extract or combination of extracts of Holothuria mexicana. In some embodiments, the biomass composition comprises from 40% to 50% of a visceral extract, gonad extract, testes extract, ovary extract or combination of extracts of Holothuria mexicana. In some embodiments, the biomass composition comprises about 40% of a visceral extract, gonad extract, testes extract, ovary extract or combination of extracts of Holothuria mexicana. In some embodiments, the biomass composition comprises about 45% of a visceral extract, gonad extract, testes extract, ovary extract or combination of extracts of Holothuria mexicana. In some embodiments, the biomass composition comprises an extract from the whole body of Holothuria mexicana.

In some embodiments, the biomass composition comprises from 1% to 99% of a visceral extract, gonad extract, testes extract, ovary extract or combination of extracts of Holothuria californicus. In some embodiments, the biomass composition comprises from 10% to 99% of a visceral extract, gonad extract, testes extract, ovary extract or combination of extracts of Holothuria californicus. In some embodiments, the biomass composition comprises from 20% to 99% of a visceral extract, gonad extract, testes extract, ovary extract or combination of extracts of Holothuria californicus. In some embodiments, the biomass composition comprises from 40% to 99% of a visceral extract, gonad extract, testes extract, ovary extract or combination of extracts of Holothuria californicus. In some embodiments, the biomass composition comprises from 20% to 80% of a visceral extract, gonad extract, testes extract, ovary extract or combination of extracts of Holothuria californicus. In some embodiments, the biomass composition comprises from 30% to 60% of a visceral extract, gonad extract, testes extract, ovary extract or combination of extracts of Holothuria californicus. In some embodiments, the biomass composition comprises from 40% to 50% of a visceral extract, gonad extract, testes extract, ovary extract or combination of extracts of Holothuria californicus. In some embodiments, the biomass composition comprises about 40% of a visceral extract, gonad extract, testes extract, ovary extract or combination of extracts of Holothuria californicus. In some embodiments, the biomass composition comprises about 45% of a visceral extract, gonad extract, testes extract, ovary extract or combination of extracts of Holothuria californicus. In some embodiments, the biomass composition comprises an extract from the whole body of Holothuria californicus.

In some embodiments, the biomass composition comprises from 1% to 99% of a visceral extract, gonad extract, testes extract, ovary extract or combination of extracts of Stichopus chlorontus. In some embodiments, the biomass composition comprises from 10% to 99% of a visceral extract, gonad extract, testes extract, ovary extract or combination of extracts of Stichopus chlorontus. In some embodiments, the biomass composition comprises from 20% to 99% of a visceral extract, gonad extract, testes extract, ovary extract or combination of extracts of Stichopus chlorontus. In some embodiments, the biomass composition comprises from 40% to 99% of a visceral extract, gonad extract, testes extract, ovary extract or combination of extracts of Stichopus chlorontus. In some embodiments, the biomass composition comprises from 60% to 99% of a visceral extract, gonad extract, testes extract, ovary extract or combination of extracts of Stichopus chlorontus. In some embodiments, the biomass composition comprises from 20% to 80% of a visceral extract, gonad extract, testes extract, ovary extract or combination of extracts of Stichopus chlorontus. In some embodiments, the biomass composition comprises from 30% to 60% of a visceral extract, gonad extract, testes extract, ovary extract or combination of extracts of Stichopus chlorontus. In some embodiments, the biomass composition comprises from 40% to 50% of a visceral extract, gonad extract, testes extract, ovary extract or combination of extracts of Stichopus chlorontus. In some embodiments, the biomass composition comprises about 40% of a visceral extract, gonad extract, testes extract, ovary extract or combination of extracts of Stichopus chlorontus. In some embodiments, the biomass composition comprises about 45% of a visceral extract, gonad extract, testes extract, ovary extract or combination of extracts of Stichopus chlorontus. In some embodiments, the biomass composition comprises an extract from the whole body of Stichopus chlorontus.

In some embodiments, the biomass composition comprises from 1% to 99% of a visceral extract, gonad extract, testes extract, ovary extract or combination of extracts of Stichopus horrens.

In some embodiments, the biomass composition comprises from 10% to 99% of a visceral extract, gonad extract, testes extract, ovary extract or combination of extracts of Stichopus horrens. In some embodiments, the biomass composition comprises from 20% to 99% of a visceral extract, gonad extract, testes extract, ovary extract or combination of extracts of Stichopus horrens. In some embodiments, the biomass composition comprises from 40% to 99% of a visceral extract, gonad extract, testes extract, ovary extract or combination of extracts of Stichopus horrens. In some embodiments, the biomass composition comprises from 60% to 99% of a visceral extract, gonad extract, testes extract, ovary extract or combination of extracts of Stichopus horrens. In some embodiments, the biomass composition comprises from 20% to 80% of a visceral extract, gonad extract, testes extract, ovary extract or combination of extracts of Stichopus horrens. In some embodiments, the biomass composition comprises from 30% to 60% of a visceral extract, gonad extract, testes extract, ovary extract or combination of extracts of Stichopus horrens. In some embodiments, the biomass composition comprises from 40% to 50% of a visceral extract, gonad extract, testes extract, ovary extract or combination of extracts of Stichopus horrens. In some embodiments, the biomass composition comprises about 40% of a visceral extract, gonad extract, testes extract, ovary extract or combination of extracts of Stichopus horrens. In some embodiments, the biomass composition comprises about 45% of a visceral extract, gonad extract, testes extract, ovary extract or combination of extracts of Stichopus horrens. In some embodiments, the biomass composition comprises an extract from the whole body of Stichopus horrens.

In some embodiments, the biomass composition comprises from 1% to 99% of a visceral extract, gonad extract, testes extract, ovary extract or combination of extracts of Stichopus japonicus. In some embodiments, the biomass composition comprises from 10% to 99% of a visceral extract, gonad extract, testes extract, ovary extract or combination of extracts of Stichopus japonicus. In some embodiments, the biomass composition comprises from 20% to 99% of a visceral extract, gonad extract, testes extract, ovary extract or combination of extracts of Stichopus japonicus. In some embodiments, the biomass composition comprises from 40% to 99% of a visceral extract, gonad extract, testes extract, ovary extract or combination of extracts of Stichopus japonicus. In some embodiments, the biomass composition comprises from 20% to 80% of a visceral extract, gonad extract, testes extract, ovary extract or combination of extracts of Stichopus japonicus. In some embodiments, the biomass composition comprises from 30% to 60% of a visceral extract, gonad extract, testes extract, ovary extract or combination of extracts of Stichopus japonicus. In some embodiments, the biomass composition comprises from 40% to 50% of a visceral extract, gonad extract, testes extract, ovary extract or combination of extracts of Stichopus japonicus. In some embodiments, the biomass composition comprises about 40% of a visceral extract, gonad extract, testes extract, ovary extract or combination of extracts of Stichopus japonicus. In some embodiments, the biomass composition comprises about 45% of a visceral extract, gonad extract, testes extract, ovary extract or combination of extracts of Stichopus japonicus. In some embodiments, the biomass composition comprises about an extract from the whole body of Stichopus japonicus.

In some embodiments, the biomass composition comprises from 1% to 99% of a visceral extract, gonad extract, testes extract, ovary extract or combination of extracts of Apostichopus japonicus. In some embodiments, the biomass composition comprises from 10% to 99% of a visceral extract, gonad extract, testes extract, ovary extract or combination of extracts of Apostichopus japonicus. In some embodiments, the biomass composition comprises from 20% to 99% of a visceral extract, gonad extract, testes extract, ovary extract or combination of extracts of Apostichopus japonicus. In some embodiments, the biomass composition comprises from 40% to 99% of a visceral extract, gonad extract, testes extract, ovary extract or combination of extracts of Apostichopus japonicus. In some embodiments, the biomass composition comprises from 20% to 80% of a visceral extract, gonad extract, testes extract, ovary extract or combination of extracts of Apostichopus japonicus. In some embodiments, the biomass composition comprises from 30% to 60% of a visceral extract, gonad extract, testes extract, ovary extract or combination of extracts of Apostichopus japonicus. In some embodiments, the biomass composition comprises from 40% to 50% of a visceral extract, gonad extract, testes extract, ovary extract or combination of extracts of Apostichopus japonicus. In some embodiments, the biomass composition comprises about 40% of a visceral extract, gonad extract, testes extract, ovary extract or combination of extracts of Apostichopus japonicus. In some embodiments, the biomass composition comprises about 45% of a visceral extract, gonad extract, testes extract, ovary extract or combination of extracts of Apostichopus japonicus. In some embodiments, the biomass composition comprises an extract from the whole body of Apostichopus japonicus.

In some embodiments, the biomass composition comprises from 1% to 99% of a visceral extract, gonad extract, testes extract, ovary extract or combination of extracts of Cucumaria japonica. In some embodiments, the biomass composition comprises from 10% to 99% of a visceral extract, gonad extract, testes extract, ovary extract or combination of extracts of Cucumaria japonica. In some embodiments, the biomass composition comprises from 20% to 99% of a visceral extract, gonad extract, testes extract, ovary extract or combination of extracts of Cucumaria japonica. In some embodiments, the biomass composition comprises from 40% to 99% of a visceral extract, gonad extract, testes extract, ovary extract or combination of extracts of Cucumaria japonica. In some embodiments, the biomass composition comprises from 20% to 80% of a visceral extract, gonad extract, testes extract, ovary extract or combination of extracts of Cucumaria japonica. In some embodiments, the biomass composition comprises from 30% to 60% of a visceral extract, gonad extract, testes extract, ovary extract or combination of extracts of Cucumaria japonica. In some embodiments, the biomass composition comprises from 40% to 50% of a visceral extract, gonad extract, testes extract, ovary extract or combination of extracts of Cucumaria japonica. In some embodiments, the biomass composition comprises about 40% of a visceral extract, gonad extract, testes extract, ovary extract or combination of extracts of Cucumaria japonica. In some embodiments, the biomass composition comprises about 45% of a visceral extract, gonad extract, testes extract, ovary extract or combination of extracts of Cucumaria japonica. In some embodiments, the biomass composition comprises an extract from the whole body of Cucumaria japonica.

### Seagrass

Provided herein the biomass composition comprise an extract from seagrass. As used herein, the term “seagrass” is used interchangeably with “seaweed.” In some embodiments, the biomass composition is about: 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or 99% of seagrass extract. In some embodiments, the majority of the biomass composition comprises one or more seagrass derivatives. In some embodiments, the biomass composition comprises an extract from seagrass. In some embodiments, the extract from seagrass comprise an extract selected from the group consisting of: Sargassum pallidum, Sargassum polycystum, Sargassum muticum, Sargassum filipendula, and Sargassum vulgare.

In some embodiments, the biomass composition comprises from 1% to 99% of a whole plant extract, blade extract, stipe extract, holdfast extract, or combination of extracts of Sargassum pallidum. In some embodiments, the biomass composition comprise from 1% to 80% of a whole plant extract, blade extract, stipe extract, holdfast extract, or combination of extracts of Sargassum pallidum. In some embodiments, the biomass composition comprise from 1% to 60% of a whole plant extract, blade extract, stipe extract, holdfast extract, or combination of extracts of Sargassum pallidum. In some embodiments, the biomass composition comprise from 1% to 50% of a whole plant extract, blade extract, stipe extract, holdfast extract, or combination of extracts of Sargassum pallidum. In some embodiments, the biomass composition comprise from 1% to 40% of a whole plant extract, blade extract, stipe extract, holdfast extract, or combination of extracts of Sargassum pallidum. In some embodiments, the biomass composition comprise from 1% to 30% of a whole plant extract, blade extract, stipe extract, holdfast extract, or combination of extracts of Sargassum pallidum. In some embodiments, the biomass composition comprise from 1% to 20% of a whole plant extract, blade extract, stipe extract, holdfast extract, or combination of extracts of Sargassum pallidum. In some embodiments, the biomass composition comprise from 1% to 10% of a whole plant extract, blade extract, stipe extract, holdfast extract, or combination of extracts of Sargassum pallidum. In some embodiments, the biomass composition comprise from 1% to 5% of a whole plant extract, blade extract, stipe extract, holdfast extract, or combination of extracts of Sargassum pallidum. In some embodiments, the biomass composition comprises about 5% of a whole plant extract, blade extract, stipe extract, holdfast extract, or combination of extracts of Sargassum pallidum. In some embodiments, the biomass composition comprises an extract from the whole body of Sargassum pallidum.

In some embodiments, the biomass composition comprise from 1% to 99% of a whole plant extract, blade extract, stipe extract, holdfast extract, or combination of extracts of Sargassum polycystum. In some embodiments, the biomass composition comprise from 10% to 80% of a whole plant extract, blade extract, stipe extract, holdfast extract, or combination of extracts of Sargassum polycystum. In some embodiments, the biomass composition comprise from 10% to 60% of a whole plant extract, blade extract, stipe extract, holdfast extract, or combination of extracts of Sargassum polycystum. In some embodiments, the biomass composition comprise from 1% to 50% of a whole plant extract, blade extract, stipe extract, holdfast extract, or combination of extracts of Sargassum polycystum. In some embodiments, the biomass composition comprise from 10% to 40% of a whole plant extract, blade extract, stipe extract, holdfast extract, or combination of extracts of Sargassum polycystum. In some embodiments, the biomass composition comprise from 1% to 30% of a whole plant extract, blade extract, stipe extract, holdfast extract, or combination of extracts of Sargassum polycystum. In some embodiments, the biomass composition comprise from 1% to 20% of a whole plant extract, blade extract, stipe extract, holdfast extract, or combination of extracts of Sargassum polycystum. In some embodiments, the biomass composition comprise from 1% to 10% of a whole plant extract, blade extract, stipe extract, holdfast extract, or combination of extracts of Sargassum polycystum. In some embodiments, the biomass composition comprise from 1% to 5% of a whole plant extract, blade extract, stipe extract, holdfast extract, or combination of extracts of Sargassum polycystum. In some embodiments, the biomass composition comprises about 5% of a whole plant extract, blade extract, stipe extract, holdfast extract, or combination of extracts of Sargassum polycystum. In some embodiments, the biomass composition comprises an extract from the whole body of Sargassum polycystum.

### Sea Squirt

Provided herein the biomass composition comprise an extract from sea squirt. In some embodiments, the biomass composition is about: 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or 99% of sea squirt extract. In some embodiments, the majority of the biomass composition comprises one or more sea squirt derivatives. In some embodiments, the biomass composition comprises an extract from sea squirt. In some embodiments, the extract from sea squirt comprise an extract selected from the group consisting of: Ecteinascidia turbinata, Styela clava, Ciona intestinalis, and Ascidiella aspersa.

In some embodiments, the biomass composition comprises from 1% to 99% of a visceral extract, body wall extract, gonad extract, testes extract, ovary extract or combination of extracts of Ecteinascidia turbinata. In some embodiments, the biomass composition comprises from 1% to 80% of a visceral extract, body wall extract, gonad extract, testes extract, ovary extract or combination of extracts of Ecteinascidia turbinata. In some embodiments, the biomass composition comprises from 1% to 60% of a visceral extract, body wall extract, gonad extract, testes extract, ovary extract or combination of extracts of Ecteinascidia turbinata. In some embodiments, the biomass composition comprises from 1% to 50% of a visceral extract, body wall extract, gonad extract, testes extract, ovary extract or combination of extracts of Ecteinascidia turbinata. In some embodiments, the biomass composition comprises from 1% to 40% of a visceral extract, body wall extract, gonad extract, testes extract, ovary extract or combination of extracts of Ecteinascidia turbinata. In some embodiments, the biomass composition comprises from 1% to 30% of a visceral extract, body wall extract, gonad extract, testes extract, ovary extract or combination of extracts Ecteinascidia turbinata. In some embodiments, the biomass composition comprises from 1% to 20% of a visceral extract, body wall extract, gonad extract, testes extract, ovary extract or combination of extracts of Ecteinascidia turbinata. In some embodiments, the biomass composition comprises from 1% to 10% of a visceral extract, body wall extract, gonad extract, testes extract, ovary extract or combination of extracts of Ecteinascidia turbinata. In some embodiments, the biomass composition comprises from 1% to 5% of a visceral extract, body wall extract, gonad extract, testes extract, ovary extract or combination of extracts of Ecteinascidia turbinata. In some embodiments, the biomass composition comprises about 5% of a visceral extract, body wall extract, gonad extract, testes extract, ovary extract or combination of extracts of Ecteinascidia turbinata. In some embodiments, the biomass composition comprises an extract from the whole body of Ecteinascidia turbinata.

In some embodiments, the biomass composition comprises from 1% to 99% of a visceral extract, body wall extract, gonad extract, testes extract, ovary extract or combination of extracts of Styela clava. In some embodiments, the biomass composition comprises from 1% to 80% of a visceral extract, body wall extract, gonad extract, testes extract, ovary extract or combination of extracts of Styela clava. In some embodiments, the biomass composition comprises from 1% to 60% of a visceral extract, body wall extract, gonad extract, testes extract, ovary extract or combination of extracts of Styela clava. In some embodiments, the biomass composition comprises from 1% to 50% of a visceral extract, body wall extract, gonad extract, testes extract, ovary extract or combination of extracts of Styela clava. In some embodiments, the biomass composition comprises from 1% to 40% of a visceral extract, body wall extract, gonad extract, testes extract, ovary extract or combination of extracts of Styela clava. In some embodiments, the biomass composition comprises from 1% to 30% of a visceral extract, body wall extract, gonad extract, testes extract, ovary extract or combination of extracts Styela clava. In some embodiments, the biomass composition comprises from 1% to 20% of a visceral extract, body wall extract, gonad extract, testes extract, ovary extract or combination of extracts of Styela clava. In some embodiments, the biomass composition comprises from 1% to 10% of a visceral extract, body wall extract, gonad extract, testes extract, ovary extract or combination of extracts of Styela clava. In some embodiments, the biomass composition comprises from 1% to 5% of a visceral extract, body wall extract, gonad extract, testes extract, ovary extract or combination of extracts of Styela clava.

In some embodiments, the biomass composition comprises about 5% of a visceral extract, body wall extract, gonad extract, testes extract, ovary extract or combination of extracts of Styela clava.

In some embodiments, the biomass composition comprises an extract from the whole body of Styela clava.

In some embodiments, the biomass composition comprises from 1% to 99% of a visceral extract, body wall extract, gonad extract, testes extract, ovary extract or combination of extracts of Ciona intestinalis. In some embodiments, the biomass composition comprises from 1% to 80% of a visceral extract, body wall extract, gonad extract, testes extract, ovary extract or combination of extracts of Ciona intestinalis. In some embodiments, the biomass composition comprises from 1% to 60% of a visceral extract, body wall extract, gonad extract, testes extract, ovary extract or combination of extracts of Ciona intestinalis. In some embodiments, the biomass composition comprises from 1% to 50% of a visceral extract, body wall extract, gonad extract, testes extract, ovary extract or combination of extracts of Ciona intestinalis. In some embodiments, the biomass composition comprises from 1% to 40% of a visceral extract, body wall extract, gonad extract, testes extract, ovary extract or combination of extracts of Ciona intestinalis. In some embodiments, the biomass composition comprises from 1% to 30% of a visceral extract, body wall extract, gonad extract, testes extract, ovary extract or combination of extracts Ciona intestinalis. In some embodiments, the biomass composition comprises from 1% to 20% of a visceral extract, body wall extract, gonad extract, testes extract, ovary extract or combination of extracts of Ciona intestinalis.

In some embodiments, the biomass composition comprises from 1% to 10% of a visceral extract, body wall extract, gonad extract, testes extract, ovary extract or combination of extracts of Ciona intestinalis. In some embodiments, the biomass composition comprises from 1% to 5% of a visceral extract, body wall extract, gonad extract, testes extract, ovary extract or combination of extracts of Ciona intestinalis. In some embodiments, the biomass composition comprises about 5% of a visceral extract, body wall extract, gonad extract, testes extract, ovary extract or combination of extracts of Ciona intestinalis. In some embodiments, the biomass composition comprises an extract from the whole body of Ciona intestinalis.

In some embodiments, the biomass composition comprises from 1% to 99% of a visceral extract, body wall extract, gonad extract, testes extract, ovary extract or combination of extracts of Ascidiella aspersa. In some embodiments, the biomass composition comprises from 1% to 80% of a visceral extract, body wall extract, gonad extract, testes extract, ovary extract or combination of extracts of Ascidiella aspersa. In some embodiments, the biomass composition comprises from 1% to 60% of a visceral extract, body wall extract, gonad extract, testes extract, ovary extract or combination of extracts of Ascidiella aspersa. In some embodiments, the biomass composition comprises from 1% to 50% of a visceral extract, body wall extract, gonad extract, testes extract, ovary extract or combination of extracts of Ascidiella aspersa. In some embodiments, the biomass composition comprises from 1% to 40% of a visceral extract, body wall extract, gonad extract, testes extract, ovary extract or combination of extracts of Ascidiella aspersa. In some embodiments, the biomass composition comprises from 1% to 30% of a visceral extract, body wall extract, gonad extract, testes extract, ovary extract or combination of extracts Ascidiella aspersa.

In some embodiments, the biomass composition comprises from 1% to 20% of a visceral extract, body wall extract, gonad extract, testes extract, ovary extract or combination of extracts of Ascidiella aspersa. In some embodiments, the biomass composition comprises from 1% to 10% of a visceral extract, body wall extract, gonad extract, testes extract, ovary extract or combination of extracts of Ascidiella aspersa. In some embodiments, the biomass composition comprises from 1% to 5% of a visceral extract, body wall extract, gonad extract, testes extract, ovary extract or combination of extracts of Ascidiella aspersa. In some embodiments, the biomass composition comprises about 5% of a visceral extract, body wall extract, gonad extract, testes extract, ovary extract or combination of extracts of Ascidiella aspersa. In some embodiments, the biomass composition comprises an extract from the whole body of Ascidiella aspersa.

### Sea Urchin

Provided herein the biomass composition comprise an extract from sea urchin. In some embodiments, the biomass composition is about: 1%, 2%, 3%, 4%, 5%, 6%, 7%, 8%, 9%, 10%, 15%, 20%, 25%, 30%, 35%, 40%, 45%, 50%, 55% 60%, 65, 70%, 75%, 80%, 85, 90%, 95% or 99% of sea urchin extract. In some embodiments, the majority of the biomass composition comprises one or more sea urchin derivatives. In some embodiments, the biomass composition comprises an extract from sea urchin. In some embodiments, the extract from sea urchin comprise an extract selected from the group consisting of Holopneustes pycnotilus, Heliocidaris erythrogramma, Heliocidaris tuberculata, and Centrostephanus tenuispinus.

In some embodiments, the biomass composition comprises from 1% to 99% of a primary spine extract, secondary spine extract, tube feet extract, teeth extract, interambulacral area extract or combination of extracts of Holopneustes pycnotilus. In some embodiments, the biomass composition comprises from 1% to 80% of a primary spine extract, secondary spine extract, tube feet extract, teeth extract, interambulacral area extract or combination of extracts of Holopneustes pycnotilus. In some embodiments, the biomass composition comprises from 1% to 60% of a primary spine extract, secondary spine extract, tube feet extract, teeth extract, interambulacral area extract or combination of extracts of Holopneustes pycnotilus. In some embodiments, the biomass composition comprises from 1% to 50% of a primary spine extract, secondary spine extract, tube feet extract, teeth extract, interambulacral area extract or combination of extracts of Holopneustes pycnotilus. In some embodiments, the biomass composition comprises from 1% to 40% of a primary spine extract, secondary spine extract, tube feet extract, teeth extract, interambulacral area extract or combination of extracts of Holopneustes pycnotilus. In some embodiments, the biomass composition comprises from 1% to 30% of a primary spine extract, secondary spine extract, tube feet extract, teeth extract, interambulacral area extract or combination of extracts of Holopneustes pycnotilus. In some embodiments, the biomass composition comprises from 1% to 20% of a primary spine extract, secondary spine extract, tube feet extract, teeth extract, interambulacral area extract or combination of extracts of Holopneustes pycnotilus. In some embodiments, the biomass composition comprises from 1% to 10% of a primary spine extract, secondary spine extract, tube feet extract, teeth extract, interambulacral area extract or combination of extracts of Holopneustes pycnotilus. In some embodiments, the biomass composition comprises from 1% to 5% of a primary spine extract, secondary spine extract, tube feet extract, teeth extract, interambulacral area extract or combination of extracts of Holopneustes pycnotilus. In some embodiments, the biomass composition comprises about 5% of a primary spine extract, secondary spine extract, tube feet extract, teeth extract, interambulacral area extract or combination of extracts of Holopneustes pycnotilus. In some embodiments, the biomass composition comprises an extract from the whole body of Holopneustes pycnotilus.

In some embodiments, the biomass composition comprises from 1% to 99% of a primary spine extract, secondary spine extract, tube feet extract, teeth extract, interambulacral area extract or combination of extracts of Heliocidaris erythrogramma. In some embodiments, the biomass composition comprises from 1% to 80% of a primary spine extract, secondary spine extract, tube feet extract, teeth extract, interambulacral area extract or combination of extracts of Heliocidaris erythrogramma. In some embodiments, the biomass composition comprises from 1% to 60% of a primary spine extract, secondary spine extract, tube feet extract, teeth extract, interambulacral area extract or combination of extracts of Heliocidaris erythrogramma. In some embodiments, the biomass composition comprises from 1% to 50% of a primary spine extract, secondary spine extract, tube feet extract, teeth extract, interambulacral area extract or combination of extracts of Heliocidaris erythrogramma. In some embodiments, the biomass composition comprises from 1% to 40% of a primary spine extract, secondary spine extract, tube feet extract, teeth extract, interambulacral area extract or combination of extracts of Heliocidaris erythrogramma. In some embodiments, the biomass composition comprises from 1% to 30% of a primary spine extract, secondary spine extract, tube feet extract, teeth extract, interambulacral area extract or combination of extracts of Heliocidaris erythrogramma. In some embodiments, the biomass composition comprises from 1% to 20% of a primary spine extract, secondary spine extract, tube feet extract, teeth extract, interambulacral area extract or combination of extracts of Heliocidaris erythrogramma. In some embodiments, the biomass composition comprises from 1% to 10% of a primary spine extract, secondary spine extract, tube feet extract, teeth extract, interambulacral area extract or combination of extracts of Heliocidaris erythrogramma. In some embodiments, the biomass composition comprises from 10% to 5% of a primary spine extract, secondary spine extract, tube feet extract, teeth extract, interambulacral area extract or combination of extracts of Heliocidaris erythrogramma. In some embodiments, the biomass composition comprises about 5% of a primary spine extract, secondary spine extract, tube feet extract, teeth extract, interambulacral area extract or combination of extracts of Heliocidaris erythrogramma. In some embodiments, the biomass composition comprises an extract from the whole body of Heliocidaris erythrogramma.

In some embodiments, the biomass composition comprises from 1% to 99% of a primary spine extract, secondary spine extract, tube feet extract, teeth extract, interambulacral area extract or combination of extracts of Heliocidaris tuberculata. In some embodiments, the biomass composition comprises from 1% to 80% of a primary spine extract, secondary spine extract, tube feet extract, teeth extract, interambulacral area extract or combination of extracts of Heliocidaris tuberculata. In some embodiments, the biomass composition comprises from 1% to 60% of a primary spine extract, secondary spine extract, tube feet extract, teeth extract, interambulacral area extract or combination of extracts of Heliocidaris tuberculata. In some embodiments, the biomass composition comprises from 1% to 50% of a primary spine extract, secondary spine extract, tube feet extract, teeth extract, interambulacral area extract or combination of extracts of Heliocidaris tuberculata. In some embodiments, the biomass composition comprises from 1% to 40% of a primary spine extract, secondary spine extract, tube feet extract, teeth extract, interambulacral area extract or combination of extracts of Heliocidaris tuberculata. In some embodiments, the biomass composition comprises from 1% to 30% of a primary spine extract, secondary spine extract, tube feet extract, teeth extract, interambulacral area extract or combination of extracts of Heliocidaris tuberculata. In some embodiments, the biomass composition comprises from 1% to 20% of a primary spine extract, secondary spine extract, tube feet extract, teeth extract, interambulacral area extract or combination of extracts of Heliocidaris tuberculata. In some embodiments, the biomass composition comprises from 1% to 10% of a primary spine extract, secondary spine extract, tube feet extract, teeth extract, interambulacral area extract or combination of extracts of Heliocidaris tuberculata. In some embodiments, the biomass composition comprises from 1% to 5% of a primary spine extract, secondary spine extract, tube feet extract, teeth extract, interambulacral area extract or combination of extracts of Heliocidaris tuberculata. In some embodiments, the biomass composition comprises about 5% of a primary spine extract, secondary spine extract, tube feet extract, teeth extract, interambulacral area extract or combination of extracts of Heliocidaris tuberculata. In some embodiments, the biomass composition comprises an extract from the whole body of Heliocidaris tuberculata.

In some embodiments, the biomass composition comprises from 1% to 99% of a primary spine extract, secondary spine extract, tube feet extract, teeth extract, interambulacral area extract or combination of extracts of Centrostephanus tenuispinus. In some embodiments, the biomass composition comprises from 1% to 80% of a primary spine extract, secondary spine extract, tube feet extract, teeth extract, interambulacral area extract or combination of extracts of Centrostephanus tenuispinus. In some embodiments, the biomass composition comprises from 1% to 60% of a primary spine extract, secondary spine extract, tube feet extract, teeth extract, interambulacral area extract or combination of extracts of Centrostephanus tenuispinus. In some embodiments, the biomass composition comprises from 1% to 50% of a primary spine extract, secondary spine extract, tube feet extract, teeth extract, interambulacral area extract or combination of extracts of Centrostephanus tenuispinus. In some embodiments, the biomass composition comprises from 1% to 40% of a primary spine extract, secondary spine extract, tube feet extract, teeth extract, interambulacral area extract or combination of extracts of Centrostephanus tenuispinus. In some embodiments, the biomass composition comprises from 1% to 30% of a primary spine extract, secondary spine extract, tube feet extract, teeth extract, interambulacral area extract or combination of extracts of Centrostephanus tenuispinus. In some embodiments, the biomass composition comprises from 1% to 20% of a primary spine extract, secondary spine extract, tube feet extract, teeth extract, interambulacral area extract or combination of extracts of Centrostephanus tenuispinus. In some embodiments, the biomass composition comprises from 1% to 10% of a primary spine extract, secondary spine extract, tube feet extract, teeth extract, interambulacral area extract or combination of extracts of Centrostephanus tenuispinus. In some embodiments, the biomass composition comprises from 1% to 5% of a primary spine extract, secondary spine extract, tube feet extract, teeth extract, interambulacral area extract or combination of extracts of Centrostephanus tenuispinus. In some embodiments, the biomass composition comprises about 5% of a primary spine extract, secondary spine extract, tube feet extract, teeth extract, interambulacral area extract or combination of extracts of Centrostephanus tenuispinus. In some embodiments, the biomass composition comprises an extract from the whole body of Centrostephanus tenuispinus

Provided here is a biomass combination composition comprising biomass material from one or more marine organisms. In some embodiments, the one or more marine organisms comprise a marine invertebrate. In some embodiments, the one or more marine organisms comprise a plurality of marine invertebrates. In some embodiments, the biomass composition comprises a majority sea cucumber (MSC) blend and a smaller fraction of other marine-derived species.

Various bioactive compounds that possess immunomodulatory capabilities and anticancer properties have been identified within each of the contained marine species. Specifically, it is composed of extracts derived from two separate species of sea cucumber, seagrass, sea squirt, and sea urchin.

### Formulation A

In some embodiments, the biomass composition is formulation A, where formulation A comprises extracts from: two separate sea cucumber species, seagrass, sea squirt and sea urchin according to Table 1. In some embodiments, the two separate sea cucumber species is Holothuria nobilis and Holothuria scabra. In some embodiments, the seagrass is Sargassum pallidum. In some embodiments, the sea squirt is Steyla clava. In some embodiments, the sea urchin is Heliocidaris erythrogramma.

In some embodiments, formulation A comprises a mixture of biomass material from 1% w/w to about 50% w/w Holothuria nobilis. In some embodiments, formulation A comprises from about: 10% w/w to 2% w/w, 10% w/w to 5% w/w, 10% w/w to 10% w/w, 10% w/w to 150% w/w, 10% w/w to 20% w/w, 1% w/w to 25% w/w, 1% w/w to 30% w/w, 1% w/w to 40% w/w, 1% w/w to 50% w/w, 2% w/w to 5% w/w, 2% w/w to 10% w/w, 2% w/w to 15% w/w, 2% w/w to 20% w/w, 2% w/w to 25% w/w, 2% w/w to 30% w/w, 2% w/w to 35% w/w, 2% w/w to 40% w/w, 5% w/w to 10% w/w, 5% w/w to 15% w/w, 5% w/w to 20% w/w, 5% w/w to 25% w/w, 5% w/w to 30% w/w, 5% w/w to 35% w/w, 5% w/w to 40% w/w, 5% w/w to 45% w/w, 5% w/w to 50% w/w, 10% w/w to 15% w/w, 10% w/w to 20% w/w, 10% w/w to 25% w/w, 10% w/w to 30% w/w, 10% w/w to 35% w/w, 10% w/w to 40% w/w, 10% w/w to 45% w/w, 10% w/w to 50% w/w, 15% w/w to 20% w/w, 15% w/w to 25% w/w, 15% w/w to 30% w/w, 15% w/w to 35% w/w, 15% w/w to 40% w/w, 15% w/w to 45% w/w, 15% w/w to 50% w/w, 20% w/w to 25% w/w, 20% w/w to 30% w/w, 20% w/w to 35% w/w, 20% w/w to 40% w/w, 20% w/w to 45% w/w, 20% w/w to 50% w/w, 25% w/w to 30% w/w, 30% w/w to 35% w/w, 35% w/w to 40% w/w, 40% w/w to 45% w/w, 40% w/w to 50% w/w, or 45% w/w to 50% w/w Holothuria nobilis. In some embodiments, formulation A comprises 45% w/w of Holothuria nobilis.

In some embodiments, formulation A comprises a mixture of biomass material from 1% w/w to about 40% w/w Holothuria scabra. In some embodiments, formulation A comprises from about: 1% w/w to 2% w/w, 1% w/w to 5% w/w, 1% w/w to 10% w/w, 1% w/w to 150% w/w, 1% w/w to 20% w/w, 1% w/w to 25% w/w, 1% w/w to 30% w/w, 1% w/w to 40% w/w, 2% w/w to 5% w/w, 2% w/w to 10% w/w, 2% w/w to 15% w/w, 2% w/w to 20% w/w, 2% w/w to 25% w/w, 2% w/w to 30% w/w, 2% w/w to 35% w/w, 2% w/w to 40% w/w, 5% w/w to 10% w/w, 5% w/w to 15% w/w, 5% w/w to 20% w/w, 5% w/w to 25% w/w, 5% w/w to 30% w/w, 5% w/w to 35% w/w, 5% w/w to 40% w/w, 10% w/w to 15% w/w, 10% w/w to 20% w/w, 10% w/w to 25% w/w, 10% w/w to 30% w/w, 10% w/w to 35% w/w, 10% w/w to 40% w/w, 15% w/w to 20% w/w, 15% w/w to 25% w/w, 15% w/w to 30% w/w, 15% w/w to 35% w/w, 15% w/w to 40% w/w, 20% w/w to 25% w/w, 20% w/w to 30% w/w, 20% w/w to 35% w/w, 20% w/w to 40% w/w, 20% w/w to 45% w/w, 20% w/w to 50% w/w, 25% w/w to 30% w/w, 30% w/w to 35% w/w, or 35% w/w to 40% w/w Holothuria scabra. In some embodiments, formulation A comprises 40% w/w of Holothuria scabra.

In some embodiments, formulation A comprises a mixture of biomass material from 1% w/w to about 10% w/w Sargassum pallidum. In some embodiments, formulation A comprises from about: 1% w/w to 2% w/w, 1% w/w to 3% w/w, 1% w/w to 4% w/w, 1% w/w to 5% w/w, 1% w/w to 6% w/w, 10% w/w to 7% w/w, 10% w/w to 8% w/w, 10% w/w to 9% w/w, 10% w/w to 10% w/w, 2% w/w to 5% w/w, 2% w/w to 6% w/w, 2% w/w to 7% w/w, 2% w/w to 8% w/w, 2% w/w to 9% w/w, 2% w/w to 10% w/w, 3% w/w to 5% w/w, 4% w/w to 5% w/w, or 5% w/w to 10% w/w Sargassum pallidum. In some embodiments, formulation A comprises 5% w/w of Sargassum pallidum.

In some embodiments, formulation A comprises a mixture of biomass material from 1% w/w to about 10% w/w Heliocidaris erythrogramma. The formulation comprising biomass composition comprise about 1% w/w to 2% w/w, 1% w/w to 3% w/w, 1% w/w to 4% w/w, 1% w/w to 5% w/w, 1% w/w to 6% w/w, 1% w/w to 7% w/w, 1% w/w to 8% w/w, 1% w/w to 9% w/w, 1% w/w to 10% w/w, 2% w/w to 5% w/w, 2% w/w to 6% w/w, 2% w/w to 7% w/w, 2% w/w to 8% w/w, 2% w/w to 9% w/w, 2% w/w to 10% w/w, 3% w/w to 5% w/w, 4% w/w to 5% w/w, or 5% w/w to 10% w/w Heliocidaris erythrogramma. In some embodiments, formulation A comprises 5% w/w of Sargassum pallidum.

In some embodiments, formulation A comprises a mixture of biomass material from 1% w/w to about 10% w/w Styela clava. The formulation comprising biomass composition comprise about 10% w/w to 2% w/w, 10% w/w to 3% w/w, 10% w/w to 4% w/w, 10% w/w to 5% w/w, 10% w/w to 6% w/w, 10% w/w to 7% w/w, 10% w/w to 8% w/w, 10% w/w to 9% w/w, 10% w/w to 10% w/w, 2% w/w to 5% w/w, 2% w/w to 6% w/w, 2% w/w to 7% w/w, 2% w/w to 8% w/w, 2% w/w to 9% w/w, 2% w/w to 10% w/w, 3% w/w to 5% w/w, 4% w/w to 5% w/w, or 5% w/w to 10% w/w Styela clava. In some embodiments, formulation A comprises 5% w/w of Styela clava.

TABLE 1
Exemplary composition - Formulation A

| Name | Phylum | Class | Species | % w/w |
|---|---|---|---|---|
| Sea Cucumber | Echinodermata | Holothuria | Holothuria nobilis | 45 |
| | Echinodermata | Holothuria | Holothuria scabra | 40 |
| Seagrass | n/a | Phaephyceau | Sargassum pallidum | 5 |
| Sea Squirt | Chordata | Ascidiacea | Styela clava | 5 |
| Sea Urchin | Echinodermata | Echinoidea | Heliocidaris erythrogramma | 5 |

In some embodiments, the biomass composition comprises a suspension. In some embodiments, the suspension is a homogenous suspension. In some embodiments, the biomass composition comprises a suspension for oral administration. In some embodiments, the biomass composition comprises a liquid for oral administration. In some embodiments, the biomass composition comprises an emulsion for oral administration. In some embodiments, the biomass composition is unfiltered prior to the administering to the subject. In some embodiments, the biomass composition is filtered prior to the administering to the subject. In some embodiments, the biomass composition is not centrifuged prior to the administering to the subject. In some embodiments, the biomass composition is centrifuged prior to the administering to the subject. In some embodiments, the biomass composition further comprises a solubilizing agent prior to the administering. In some embodiments, the solubilizing agent is dimethyl sulfoxide (DMSO). In some embodiments, the biomass composition comprises a liquid for intravenous administration. In some embodiments, the biomass composition is in a single dose unit. In some embodiments, the biomass composition is encapsulation in a packet, a satchel, a gummy, a tablet, a capsule or a pill.

In some embodiments, the sachet is a foil sachet. In some embodiments, the biomass composition is comprised in a foil sachet. In some embodiments, the foil sachet comprising the biomass composition be stored at 4° C. In some embodiments, the foil sachets comprising the biomass composition be stored at −20° C. In the embodiments, prior to the administering the foil sachet is frozen. In some embodiments, prior to the administering the foil sachet is thawed. In some embodiments, a single foil sachet comprises from about 1 to about 40 ml of a suspension comprising the biomass composition. In some embodiments, a single foil sachet comprises from about 1 to about 35 ml of a suspension comprising the biomass composition. In some embodiments, a single foil sachet comprises from about 1 to about 30 ml of a suspension comprising the biomass composition. In some embodiments, a single foil sachet comprises from about 1 to about 25 ml of a suspension comprising the biomass composition. In some embodiments, a single foil sachet comprises from about 1 to about 20 ml of a suspension comprising the biomass composition. In some embodiments, a single foil sachet comprises from about 5 to about 40 ml of a suspension comprising the biomass composition. In some embodiments, a single foil sachet comprises from about 1 to about 35 ml of a suspension comprising the biomass composition. In some embodiments, a single foil sachet comprises from about 5 to about 30 ml of a suspension comprising the biomass composition. In some embodiments, a single foil sachet comprises from about 5 to about 25 ml of a suspension comprising the biomass composition. In some embodiments, a single foil sachet comprises from about 5 to about 20 ml of a suspension comprising the biomass composition. In some embodiments, a single foil sachet comprises from about 1 to about 40 ml of a suspension comprising the biomass composition. In some embodiments, a single foil sachet comprises from about 10 to about 35 ml of a suspension comprising the biomass composition. In some embodiments, a single foil sachet comprises from about 10 to about 30 ml of a suspension comprising the biomass composition. In some embodiments, a single foil sachet comprises from about 10 to about 25 ml of a suspension comprising the biomass composition. In some embodiments, a single foil sachet comprises from about 10 to about 20 ml of a suspension comprising the biomass composition. In some embodiments, a single foil sachet comprises from about 1 to about 30 g of a suspension comprising the biomass composition. In some embodiments, a single foil sachet comprises from about 10 g to about 25 g of a suspension comprising the biomass composition. In some embodiments, a single foil sachet comprises from about 20 g to about 25 g of a suspension comprising the biomass composition. In some embodiments, a single foil sachet comprises from about 20 g to about 22 g of a suspension comprising the biomass composition. In some embodiments, a single foil sachet comprises 20 g of a suspension comprising the biomass composition.

### Dosage and Method of Administration

In some embodiments, the biomass composition described herein is provided frozen in single use foil sachet, where each foil sachet is defrosted immediately prior to use. In some embodiments, the foil sachet is placed in warm (not hot) water for 20 minutes. In some embodiments, while the foil sachet is in warm water it is massaged twice during the defrosting period to aid in defrosting. Alternatively, in some embodiments, the foil sachet is placed on clean surface for 1 hour at room temperature. In some embodiments, while the foil sachet is at room temperature it is massaged 2-4 times during the hour of defrosting. In further embodiments, once the foil sachet comprising the biomass composition is defrosted, the foil sachet is then added to 1 to about 8 ounces of liquid. In some embodiments, the foil sachet is added to about 1 ounce, about 2 ounces, about 3 ounces, about 4 ounces, about 5 ounces, about 6 ounces, about 7 ounces, or about 8 ounces of liquid. In some embodiments, the liquid comprises water, juice, or any non-carbonated beverage. In some embodiments, the liquid does not comprise a carbonated beverage. In some embodiments, the liquid comprising the thawed foil sachet is lightly stirred and orally consumed by a subject in need thereof. In some embodiments, the biomass composition is in the dosage form of a solid, a semi-solid, or a liquid. In some embodiments, the biomass composition is in the dosage form of an oral formulation, in other words formulated for oral administration. In some embodiments, the biomass composition is in the dosage form of a powder, gel, capsule, tablet, or pill. In some embodiments, the biomass composition is mixed in a liquid prior to ingestion. In some embodiments, the liquid is water. In some embodiments, the biomass composition is in the dosage form of an intravenous formulation, in other words formulated for intravenous administration.

Methods and Composition for Treatment of Brain cancer

Provided herein is a method for treatment of a brain cancer in a subject in need thereof comprising administering a therapeutically effective amount of a dose of a biomass composition comprising a mixture of biomass material derived from Holothuria scabra, Holothuria nobilis, Heliocidaris erythrogramma, Styela clava, and Sargassum pallidum to the subject. In some embodiments, the brain cancer is a glioma.

In some embodiments, the glioma is glioblastoma (GBM) or diffuse intrinsic pontine glioma (DIPG). GBM remains the most common malignant primary brain neoplasm, with approximately 12,000 new cases diagnosed per year in the United States. It is considered largely incurable, with few therapeutic options. The median overall survival stands at 14.6 months, with a 5-year rate of 5.6%. The current standard of care for newly diagnosed GBM is maximal safe resection followed by concomitant radiation therapy with temozolomide (TMZ) and subsequent maintenance TMZ, with or without the addition of tumor treating fields (TTF). A dire need remains to improve outcomes in this patient population and unique sources could yield much-needed breakthroughs.

The tumor immune microenvironment (TIME) has recently been the focus of extensive study. Characterizing the nuanced histologic architecture, diverse cellular composition, and complex metabolic processes of the TIME has offered significant insight to the molecular-level features that facilitate the growth and spread of neoplastic cells. Immune cells have been identified as an important cell type in the TIME. The recruitment of immune cells into the TIME alerts the host to the presence of a tumor that it deems foreign. However, instead of interfering with the process of tumorigenesis as intended, host immune cells in the TIME is evaded, suppressed, or even exploited for carcinogenicity in response to local tumor influences. Among the immune cell components of the TIME, tumor-associated macrophages (TAMs) represent a significant subset with key functions in the promotion of tumor growth, immunomodulation, and metastasis. In the setting of GBM, these macrophages comprise up to 50% of the total number of cells in GBM. The prominence of this population is important, as studies have shown that macrophages associated with GBM may interact with glioblastoma stem cells (GSCs) and contribute to GBM growth and migration.

Targeting macrophage phenotypic shifts is especially relevant when post-treatment effects on immunomodulation are considered. Given that radiation therapy for cancer is known to promote tissue damage, which trigger macrophage phenotypic shifts, the effects of radiation on macrophage polarization have been investigated. Studies of macrophage populations in GBM have shown that following ionizing radiotherapy, the total number of macrophages decreased, and in in vivo studies have shown that the proportion of anti-inflammatory macrophages of the M2 subclass increased. M1 subclass macrophages were also more sensitive to ionizing radiation in in vitro models, potentially explaining the observed shift toward M2. Accordingly, differential stimulation of macrophage polarization depending on the dose of radiation, higher doses of radiation (>8 Gy) have been associated with a shift toward an anti-inflammatory macrophage phenotype suggesting that higher dosing of radiation, as used in the treatment of aggressive cancers like GBM, may contribute to tumorigenic activity of the irradiated zone, in part through phenotypic modulation of the macrophage populations associated with these sites. This phenomenon further demonstrates the potential for therapies, like the biomass composition described herein, that target phenotypic shifts toward PI/M1 macrophages to potentiate anti-tumor effects. Glioblastoma (GBM) is an aggressive and ultimately fatal neoplasm. Given its high rate of recurrence and mortality with current standards of care, the identification of novel therapeutic agents is critical. In the context of gliomas, higher ratios of the subclasses M2/M1 were found in GBM than in other gliomas, and were found to correlate with glioma proliferation and mortality. The concept of influencing the shift toward a PI phenotype therefore presents a potentially promising strategy for anti-cancer therapies, and several molecules with established immunomodulatory functions have demonstrated the ability to promote a shift toward an inflammatory phenotype.

In some embodiments, treatment of a brain cancer using a composition described herein provides for reduction in cancer mass. In some embodiments, treatment of a brain cancer using a composition described herein provides for reduction in cancer volume. In some embodiments, treatment of a brain cancer using a composition described herein provides for reduction in cancer size. In some embodiments, treatment of a brain cancer using a composition described in here provides for reduction in cancer recurrence. In some embodiments, treatment of a brain cancer using a composition described herein provides for reduction in cancer growth. In some embodiments, treatment of a brain cancer using a composition described herein provides for reduction in cancer recurrence. In some embodiments, treatment of a brain cancer using a composition described herein provides for reduction in cancer metastasis. In some embodiments, the brain cancer is a glioma. In some embodiments, the brain cancer is an oligodendroglioma, an ependymoma, a medulloblastoma, a choroid plexus carcinoma, a pineoblastoma, a menmigioma, an acoustic neuroma, or a craniopharyngioma. In some embodiments, treatment of a glioma using a composition described herein provides for reduction in cancer mass. In some embodiments, treatment of a glioma using a composition described herein provides for reduction in cancer volume. In some embodiments, treatment of a glioma using a composition described herein provides for reduction in cancer size. In some embodiments, treatment of a glioma using a composition described in here provides for reduction in cancer recurrence. In some embodiments, treatment of a glioma using a composition described herein provides for reduction in cancer growth. In some embodiments, treatment of a glioma using a composition described herein provides for reduction in cancer recurrence. In some embodiments, treatment of a glioma using a composition described herein provides for reduction in cancer metastasis. In some embodiments, the glioma is a glioblastoma.

Provided herein is a method for treatment of a glioma. A glioma refers to a tumor arising in the brain or spine, and is typically derived from or associated with glial cells. In some embodiments, glioma as referred to herein includes, but is not limited to, oligodendrogliomas (derived from oligodendrocytes), ependymomas (derived from ependymal cells), astrocytomas (derived from astrocytes, glioblastoma (glioblastoma multiforme or grade IV astrocytoma)), brainstem glioma (develops in the brain stem), optic nerve glioma (develops in or around the optic nerve), or mixed gliomas (such as oligoastrocytomas, containing cells from different types of glia).

In some embodiment, a glioma comprises an astrocytoma, a brain stem glioma, a diffuse intrinsic pontine glioma, an ependymoma, a glioblastoma, a mixed glioma, an oligodendroglioma, or an optic nerve glioma.

In some embodiments, the glioma is a low-grade glioma. In some embodiments, the glioma is a high-grade glioma. In some embodiments, the glioma is a grade I glioma. In some embodiments, the glioma is a grade II glioma. In some embodiments, the glioma is a grade III glioma. In some embodiments, the glioma is a grade IV glioma. In some cases, the glioma is low grade glioma, or grade II glioma. Staging or grading or cancer in general and glioma in particular is well known in the art. By means of example, glioma may be graded according to the grading system of the World Health Organization (e.g., WHO grade II oligodendroglioma). In some embodiments, the glioma is a primary glioma. In some embodiments, the glioma is a metastatic (or a secondary) glioma. In some embodiments, glioma is a recurrent glioma.

In some embodiments, the glioma is characterized by IDH1 and/or IDH2 (isocytrate dehydrogenase 1/2) mutations. In some embodiments, the IDH1 mutation is R132H. In some embodiments, the glioma is characterized by deletion of chromosome arms 1p and/or 19q. In some embodiments, the glioma is characterized by an IDH1 and an IDH2 mutations, such as IDH1 R132H mutation, and co-deletion of chromosome arms 1p and/or 19q. In some embodiments, the glioma is characterized by a CIC (capicua transcriptional repressor) mutation. In some embodiments, the glioma is characterized by IDH1 and/or IDH2 mutations, such as IDH1 R132H mutation, and CIC mutation. In some embodiments, the glioma is characterized by deletion of chromosome arms 1p and/or 19q, and CIC mutation. In some embodiments, the glioma is characterized by IDH1 and/or IDH2 mutations, such as IDH1 R132H mutation, co-deletion of chromosome arms 1p and/or 19q, and CIC mutation.

In some embodiments, the glioma is a glioblastoma. In some embodiments, the glioblastoma comprises a glioblastoma multiforme, a newly diagnosed glioblastoma, a giant cell glioblastoma, a gliosarcoma, a drug-resistant glioblastoma, a recurrent glioblastoma, a relapsed glioblastoma, or a refractory glioblastoma.

In some embodiments, the biomass compositions disclosed herein is an adjuvant therapy. Adjuvant therapy, in the broadest sense, is a treatment that enhances or increases efficacy of a primary therapy or a standard of care therapy. In some embodiments, the adjuvant therapy increases the effect of the primary therapy. In some embodiments, the primary therapy comprises a chemotherapy, an antibody therapy, a nucleic acid therapy, or a small molecule therapy. In some embodiments, the biomass composition is an adjuvant to a chemotherapy. In some embodiments, the biomass composition is an adjuvant to an antibody therapy. In some embodiments, the biomass composition is an adjuvant to a nucleic acid therapy. In some embodiments, the biomass composition is an adjuvant to a small molecule therapy. In some embodiments, the biomass composition is an adjuvant to an antibody therapy. In some embodiments, the adjuvant therapy kills cancer cells that have metastasized. In some embodiments, the adjuvant therapy is co-administered with chemotherapy after a surgery. In some embodiments, the adjuvant therapy prevents recurrence of the cancer. In some embodiments, the adjuvant therapy increases a subject's disease-free survival. The term disease free survival refers to a subject remaining alive, without the return of the cancer, for a defined period of time. In some embodiments, the defined period of time is about 1 year, about 2 years, about 3 years, about 4 years, about 5 years, about 10 years, or about 20 years. In some embodiments, administration of the adjuvant therapy increases a subject's progression free survival.

In some embodiments, the biomass compositions disclosed herein are co-administered with an additional cancer therapy to a subject in need thereof. In some embodiments, the additional cancer therapy comprises a chemotherapy or a radiation therapy. In some embodiments, the additional cancer therapy comprises a chemotherapy and a radiation therapy. In some embodiments, the chemotherapy is carmustine. In some embodiments, the chemotherapy is carmustine wafer implants. In some embodiments, the chemotherapy is lomustine. In some embodiments, the additional cancer comprises a target therapy. In some embodiments, the targeted therapy comprises bevacizumab. In some embodiments, the chemotherapy comprises administering a therapeutically effective dose of a chemotherapeutic agent. Non-limited examples of chemotherapeutic agents include adriamycin, cisplatin, cyclophosphamide, doxorubicin, paclitaxel, oxaliplatin, daunorubicin, docetaxel, mitoxantrone, digitoxin, digoxin, septasidedin, temozolomide, carmustine, bevacizumab, or lomustine. In some embodiments, the chemotherapy comprises an immunotherapeutic agent. In some embodiments, the immunotherapeutic agent comprises alemtuzumab, ofatumumab, rituximab, zevalin, brentuximab, a PD-1 inhibitor, a PD-L1 inhibitor, a CTLA-4 inhibitor, an IDO inhibitor, a CCR7 inhibitor, a OX40 inhibitor, a TIM3 inhibitor or a LAG3 inhibitor. In some embodiments, the chemotherapy comprises administering a therapeutically effective dose of a retinoic acid (retinic acid), retinoic acid derivatives, doxorubicin (doxirubicin), vinblastine (vinblastine), vincristine (vincristine), cyclophosphamide (cyclophosphosphamide), ifosfamide (ifosfamide), cisplatin, 5-fluorouracil, camptothecin derivatives, interferons, tamoxifen (tamoxifen), and paclitaxel (taxol). In certain embodiments, the anti-cancer compound is selected from the group consisting of labetane, rubus corchorifolius, disodium pamidronate, anastrozole (anastrozole), exemestane, cyclophosphamide, epirubicin, toremifene, letrozole, trastuzumab (trastuzumab), megestrol tamoxifen (megestroltaxofen), paclitaxel, docetaxel (docetaxel), capecitabine (capecitabine), goserelin acetate (goserelin acetate), or zoledronic acid (zoledronic acid). In some embodiments, the radiation therapy comprises tumor treating fields (TTF), Intensity-modulated radiation therapy (IMRT), and Image-guided radiation therapy (IGRT).

In some embodiments, the methods of treatment disclosed herein comprise administering a biomass composition disclosed herein in conjunction with chemotherapy. In some embodiments, the biomass compositions disclosed herein are co-administered with a chemotherapeutic agent. In some embodiments, the administration of the biomass composition is before, simultaneously with, or after administration of a chemotherapeutic agent. In some embodiments, the biomass compositions disclosed herein are administered before the administration of the chemotherapeutic agent. In some embodiments, the biomass compositions disclosed herein are administered simultaneously with the administration of the chemotherapeutic agent. In some embodiments, the biomass compositions disclosed herein are administered after the administration of the chemotherapeutic agent. In some embodiments, co-administration of the biomass compositions disclosed herein with a chemotherapeutic agent result in an increase in tolerance to chemotherapy.

In some embodiments, administration of the biomass composition before, simultaneously with, or after the administration of a chemotherapy results in enhanced efficacy of the chemotherapy, as compared to a level of efficacy of the chemotherapy alone. In some embodiments, the methods disclosed herein in conjunction with standard chemotherapy reduces one or more symptoms associated with chemotherapy. In some embodiments, the one or more symptoms include nausea and fatigue. In some embodiments, the methods disclosed herein in conjunction with chemotherapy reduces one or more side effects associated with chemotherapy. In some embodiments, the one or more side effects associated with chemotherapy or radiation therapy comprise low blood cell counts, fatigue, nausea, vomiting, loss of appetite, hair loss, diarrhea, constipation, sore mouth and throat, mucositis, taste and smell changes, skin conditions, eye and vision conditions, organ and nerve damage, memory, attention and other cognitive problems or infertility.

In some embodiments, the methods of treatment disclosed herein comprise administering abiomass composition disclosed herein in conjunction with temozolomide (TMZ), or a salt thereof.

In some embodiments, the biomass compositions disclosed herein are administered before the administration of TMZ. In some embodiments, the biomass compositions disclosed herein are administered simultaneously with the administration of TMZ. In some embodiments, the biomass compositions disclosed herein are administered after the administration of TMZ. In some embodiments, co-administration of the biomass compositions disclosed herein with TMZ result in an increase in tolerance to TMZ. In some embodiments, co-administration of the biomass compositions disclosed herein with TMZ result in an increase in efficacy of TMZ, as compared to a level of efficacy of TMZ alone. In some embodiments, administration of the biomass compositions disclosed herein in conjunction with TMZ reduces one or more side effects associated with TMZ treatment. In some embodiments, the one or more side effects associated with TMZ treatment comprises fatigue, nausea, vomiting, constipation, loss of appetite, amenorrhea, infection, loss of fertility, low platelet count, pneumonia, or any combination thereof.

In some embodiments, the methods of treatment disclosed herein comprise administering a biomass composition disclosed herein in conjunction with carmustine, or a salt thereof. In some embodiments, the biomass compositions disclosed herein are administered before the administration of carmustine. In some embodiments, the biomass compositions disclosed herein are administered simultaneously with the administration of carmustine. In some embodiments, the biomass compositions disclosed herein are administered after the administration of carmustine. In some embodiments, co-administration of the biomass compositions disclosed herein with carmustine result in an increase in tolerance to carmustine. In some embodiments, co-administration of the biomass compositions disclosed herein with carmustine result in an increase in efficacy of carmustine, as compared to a level of efficacy of carmustine alone. In some embodiments, administration of the biomass compositions disclosed herein in conjunction with carmustine reduces one or more side effects associated with carmustine treatment. In some embodiments, the one or more side effects associated with carmustine treatment comprises risk of infection, bruising and bleeding, low platelet count, diarrhea, nausea, vomiting, constipation, loss of appetite, skin changes, headaches, dizziness or difficulties with balance and walking, low blood pressure, fatigue, or any combination thereof.

In some embodiments, the methods of treatment disclosed herein comprise administering a biomass composition disclosed herein before implantation of a carmustine wafer implant. In some embodiments, the biomass compositions disclosed herein are administered after the implantation of the carmustine wafer implant. In some embodiments, co-administration of the biomass compositions disclosed herein in a subject with a carmustine wafer implant results in an increase in tolerance to the carmustine wafer implant. In some embodiments, co-administration of the biomass compositions disclosed herein with the carmustine wafer implant result in an increase in efficacy of the carmustine wafer implant, as compared to a level of efficacy of carmustine wafer implant alone. In some embodiments, administration of the biomass compositions disclosed herein in conjunction with the reduces one or more side effects associated with the carmustine wafer implant. In some embodiments, the one or more side effects associated with the carmustine wafer implant treatment comprises slow wound healing, swelling of the brain, difficulty with language, confusion, anxiety and depression, feeling weak and loss of movement, inflammation, feeling or being sick, constipation, hair loss, skin rash, headaches, infection, pain, seizures, difficulty sleeping or getting to sleep, abdominal pain, urinary incontinence, oral candida, eye problems including but not limited to pair, blurred, or abnormal vision, paralysis, swelling or the arms and legs, or any combination thereof.

In some embodiments, the methods of treatment disclosed herein comprise administering a biomass composition disclosed herein in conjunction with lomustine, or a salt thereof. In some embodiments, the biomass compositions disclosed herein are administered before the administration of lomustine. In some embodiments, the biomass compositions disclosed herein are administered simultaneously with the administration of lomustine. In some embodiments, the biomass compositions disclosed herein are administered after the administration of lomustine. In some embodiments, co-administration of the biomass compositions disclosed herein with lomustine result in an increase in tolerance to lomustine. In some embodiments, co-administration of the biomass compositions disclosed herein with lomustine result in an increase in efficacy of lomustine, as compared to a level of efficacy of lomustine alone. In some embodiments, administration of the biomass compositions disclosed herein in conjunction with lomustine reduces one or more side effects associated with lomustine treatment. In some embodiments, the one or more side effects associated with lomustine treatment comprises awkwardness, black tarry stools, bleeding gums, blood in the urine or stools, chest pain, confusion, cough or hoarseness, decrease in urination, fever or chills, lower back or side pain, painful or difficult urination, pale skin, pinpoint red spots on the skin, shortness of breath, slurred speech, sore throat, sores, ulcers, or white spots on the lips or in the mouth, swelling of the feet or lower legs, troubled breathing with exertion, unusual bleeding or bruising, unusual tiredness or weakness, or any combination thereof.

In some embodiments, administration of the compositions provided herein provide for a reduction in nausea, vomiting, coughing, sleeping disturbance, headaches, depression, anxiety, constipation, seizures, or any combination thereof. In some embodiments, administration of a composition described herein provides a decrease in nausea. In some embodiments, administration of a composition described herein provides a decrease in constipation. In some embodiments, administration of a composition described herein provides a decrease in diarrhea. In some embodiments, administration of a composition described herein provides for a decrease in side effects of treatment. In some embodiments, administration of a composition described herein provides for a decrease in feelings of illness. In some embodiments, administration of a composition described herein provides for a decrease in time spent in bed. In some embodiments, administration of a composition described herein provides for a reduction in bed sores. In some embodiments, administration of a composition described herein provides for a decrease in seizures.

In some embodiments, administration of a composition described herein provides for a decrease in headaches. In some embodiments, administration of a composition described herein provides for a reduction in anxiety and/or stress. In some embodiments, administration of a composition described herein provides for a reduction in sleep disturbance. In some embodiments, administration of a composition described herein provides for a reduction in insomnia. In some embodiments, administration of the compositions provided herein provide for an improvement in somnolence (ability to fall asleep), the amount and quality of sleep, energy, metabolism, concentration, memory, motor skills, motor functions, coordination, libido, or any combination thereof. In some embodiments, administration of a composition described herein provides for an increase in a subject's ability to concentrate. In some embodiments, administration of a composition described herein provides an increase in metabolism. In some embodiments, administration of a composition described herein provides for an increase in a subject's memory. In some embodiments, administration of a composition described herein provides for an increased amount of sleep. In some embodiments, administration of a composition described herein provides for an improvement in sleep quality. In some embodiments, administration of a composition described herein provides for an improvement in libido. In some embodiments, administration of a composition described herein provides for improved coordination. In some embodiments, administration of a composition described herein provides for improved motor functions.

### EMBODIMENTS

A method of treatment or reduction of a brain cancer in a subject in need thereof, the method comprising administering a therapeutically effective dose of a biomass composition comprising a mixture of biomass material derived from Holothuria scabra, Holothuria nobilis, Heliocidaris erythrogramma, Styela clava, and Sargassum pallidum to the subject.

The method of embodiment 1, wherein the brain cancer is a glioma.

The method of embodiment 2, wherein the glioma comprises an astrocytoma, a diffuse intrinsic pontine glioma, or a glioblastoma.

The method of embodiment 3, wherein the glioblastoma comprises a glioblastoma multiforme, a newly diagnosed glioblastoma, a giant cell glioblastoma, a gliosarcoma, a drug-resistant glioblastoma, a recurrent glioblastoma, a relapsed glioblastoma, or a refractory glioblastoma.

The method of embodiment 1, wherein the brain cancer is an oligodendroglioma, an ependymoma, a medulloblastoma, a choroid plexus carcinoma, a pineoblastoma, a menmigioma, an acoustic neuroma, or a craniopharyngioma.

The method of embodiment 3, wherein the astrocytoma comprises a grade I astrocytoma, a grade II astrocytoma, a grade III astrocytoma, or a grade IV astrocytoma.

The method of embodiment 6, wherein the astrocytoma is a grade IV astrocytoma.

The method of embodiment 7, wherein the grade IV astrocytoma does not comprise an isocitrate dehydrogenase 1 (IDH1) mutation.

The method of embodiment 7 wherein the grade IV astrocytoma comprises an isocitrate dehydrogenase 1 (IDH1) mutation.

The method of embodiment 1, wherein the biomass composition is administered orally.

The method of embodiment 1, wherein the biomass composition is administered intravenously.

The method of embodiment 1, wherein the administering of the therapeutically effective dose of the biomass composition comprises oral administration.

The method of embodiment 1, wherein the administering is daily or twice daily.

The method of embodiment 13, wherein the biomass composition is administered to the subject in one or two oral doses per day.

The method of embodiment 13, wherein the biomass composition is administered to the subject in one or two intravenous doses per day.

The method of any one of embodiments 1 to 15, further comprising administering to the subject an additional cancer therapy.

The method of embodiment 16, wherein the additional cancer therapy comprises chemotherapy or radiation therapy.

The method of embodiment 16, wherein the additional cancer therapy comprises chemotherapy and radiation therapy.

The method of embodiment 17 or 18, wherein the chemotherapy comprises temozolomide, carmustine, bevacizumab, or lomustine.

The method of embodiment 19, wherein the chemotherapy is temozolomide.

The method of embodiment 17 or 18, wherein the radiation therapy comprises tumor treating fields (TTF), Intensity-modulated radiation therapy (IMRT), and Image-guided radiation therapy (IGRT).

The method of any one of embodiments 1 to 21, wherein the majority of biomass material is from sea cucumber.

The method of any one of embodiments 1 to 22, wherein the biomass composition comprising the biomass material comprises an extract derived from at least two species of sea cucumber, a species of seagrass, a species of sea squirt, and a species of sea urchin.

The method of embodiment 23, wherein the extract derived from the at least two species of sea cucumber comprises an extract from Holothuria scabra and an extract from Holothuria nobilis.

The method of embodiment 24, wherein the biomass composition comprises from about 20% w/w to about 40% w/w of the extract from Holothuria scabra.

The method of embodiment 25, wherein the extract from Holothuria scabra is 40% w/w of the biomass composition.

The method of embodiment 26, wherein the biomass composition comprises from about 25% w/w to about 50% w/w of the extract from Holothuria nobilis.

The method of embodiment 27, wherein the extract from Holothuria nobilis is 45% w/w of the biomass composition.

The method of embodiment 23, wherein the extract derived from the species of seagrass comprises an extract from Sargassum pallidum.

The method of embodiment 29, wherein the biomass composition comprises from about 1% w/w to about 10% w/w of the extract from Sargassum pallidum.

The method of embodiment 30, wherein the extract from Sargassum pallidum is 5% w/w of the biomass composition.

The method of embodiment 23, wherein the extract derived from the species of sea squirt comprises an extract from Styela clava.

The method of embodiment 32, wherein the biomass composition comprises from about 1% w/w to about 10% w/w of the extract from Styela clava.

The method of embodiment 33, wherein the extract from Styela clava is 5% w/w of the biomass composition.

The method of embodiment 23, wherein the extract derived from the species of sea urchin comprises an extract from Heliocidaris erythrogramma.

The method of embodiment 35, wherein the biomass composition comprises from about 1% w/w to about 10% w/w of the extract from Heliocidaris erythrogramma.

The method of embodiment 36, wherein the extract from Heliocidaris erythrogramma is 5% w/w of the biomass composition.

The method of embodiment 23, wherein the extract derived from the at least two species of sea cucumber, the species of seagrass, the species of sea squirt, and the species of sea urchin comprises a whole-body extract.

The method of embodiment 1, wherein the biomass composition is unfiltered prior to the administering to the subject.

The method of embodiment 1, wherein the biomass composition is filtered prior to the administering to the subject.

The method of embodiment 1, wherein the biomass composition is not centrifuged prior to the administering to the subject.

The method of embodiment 1, wherein the biomass composition is centrifuged prior to the administering to the subject.

The method of embodiment 1, wherein the biomass composition further comprises a solubilizing agent.

The method of embodiment 43, wherein the solubilizing agent is dimethyl sulfoxide (DMSO).

### EXAMPLES

### Example 1: In Vitro Study

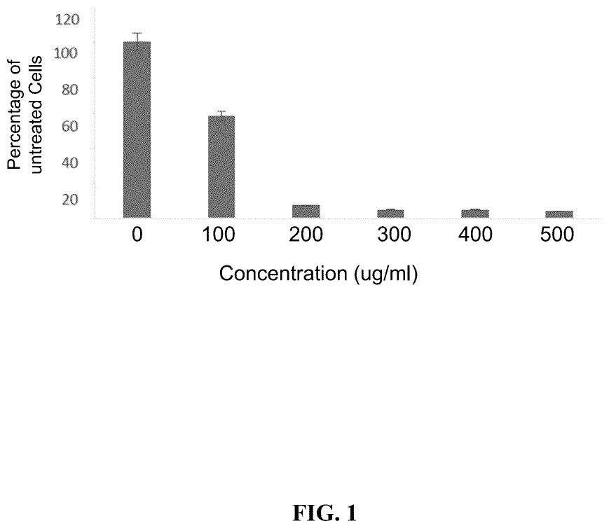

A human glioma cell line (U87) was used to assess the U87 sensitivity to the biomass composition. Cells were plated, and treated with different concentrations of the biomass compositions (0 μg/mL, 100 μg/mL, 200 μg/mL, 300 μg/mL, 400 μg/mL, 500 μg/mL). Cells were then incubated with the biomass composition for 24 hours under tissue culture conditions. Cell viability was then determined. As shown in FIG. 1, U87 cells treated with 100 μg/mL of the biomass composition had a 40% reduction in cell viability. As the concentration of the biomass composition increased, the cell viability also decreased as from 200 μg/mL less than 20% of the cells remained viable.

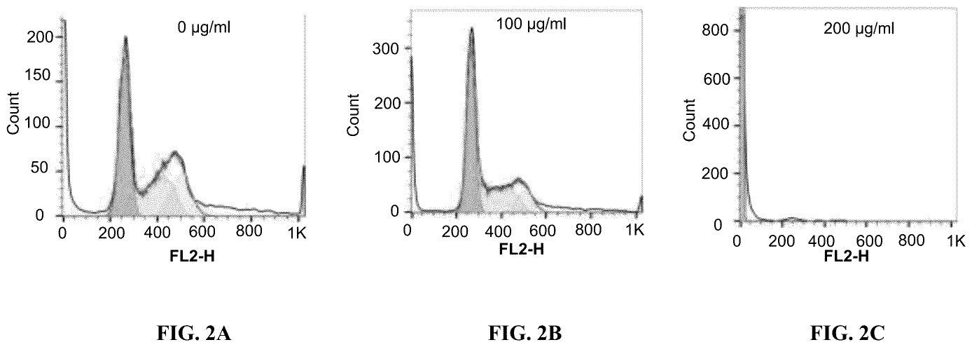

U87 cells were then treated for 24 hours with 0 μg/mL, 100 μg/mL, and 200 μg/mL of the biomass composition. After 24 hours, the cells were then stained with propidium iodide, and analyzed by flow cytometry (FIGS. 2A-2C). At 100 μg/mL, the U87 cells accumulated in S phase, represented by the second peak (FIG. 2B). While at 200 μg/mL almost all of the U87 cells were in Sub-G1 represented by the peak at the far left, which reflects apoptosis (FIG. 2C).

### Example 2: Preparation of Formulation A

For the following experiments, formulation A comprising a mixture of biomass materials from two species of sea cucumbers, a species of sea grass, a species of sea squirt, and a species of sea urchin was utilized. Formulation A as described herein comprises 45% H. nobilis, 40% H. scabra, 5% S. pallidum, 5% S. clava and 5% H. erythrogramma (Table 2). Formulation A is stored in a freezer at approximately −20° C., prior to administration formulation A was thawed at room temperature for an hour, and then lightly mixed into a non-carbonated liquid (e.g., water). Once the formulation was fully amalgamated, it was ready to be administered.

TABLE 2
Formulation A

| Name | Species | % w/w |
|---|---|---|
| Sea Cucumber | H. nobilis | 45 |
| | H. scabra | 40 |
| Seagrass | S. pallidum | 5 |
| Sea Squirt | S. clava | 5 |
| Sea Urchin | H. erythrogramma | 5 |

### Example 3: Human Study

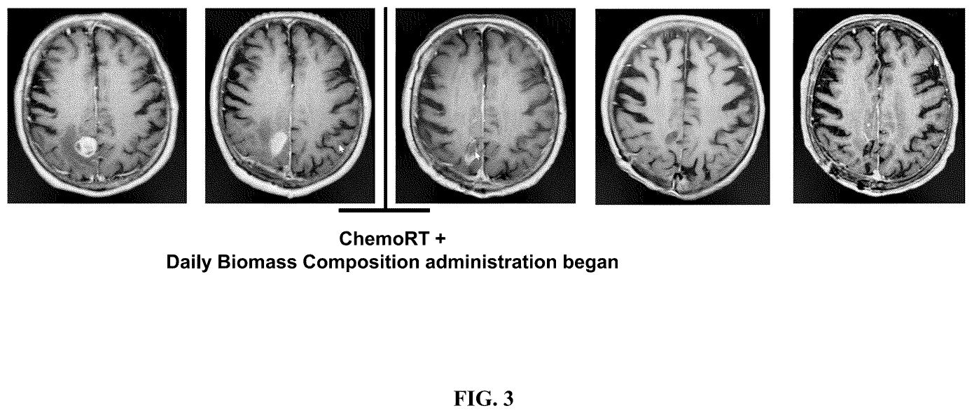

A 74-year-old female subject with a past medical history of chronic obstructive pulmonary disease, Charcot-Marie-Tooth disorder, abdominal aortic aneurysm status post repair, hypertension, and hyperlipidemia presented with a seizure-like event. Magnetic resonance imaging (MRI) with gadolinium revealed a 2.0×2.7 cm right parietal contrast-enhancing mass (FIG. 3). Shortly after neuroimaging, the subject underwent a craniotomy. Postoperative imaging indicated a gross total resection with resultant post-operative blood products within the cavity. Pathologic diagnosis on the resected specimen revealed a glioblastoma multiforme (GBM) with wild-type isocitrate dehydrogenase 1 (IDH1) and an unmethylated methylguanine methyltransferase (MGMT) promoter region.

Two weeks following the craniotomy, the subject was administered the biomass composition twice daily, at 20 mL for each dose. Two weeks after this, the subject also initiated concurrent chemotherapy and radiation therapy. The subject went on to complete a full course of chemoradiation with a total dose of 6000 cGy delivered over 6 weeks concurrent with temozolomide (TMZ) and the formulation A as disclosed herein. Once formulation was ready to be administered as described in Example 2, the subject was immediately administered the mixture through oral administration. This process was repeated 12 to 15 hours later, for a total of two doses of the biomass composition administered daily. Following the completion of chemoradiation, the subject did not undergo maintenance TMZ, but continued administering the biomass composition twice daily, as described previously. The subject remained compliant with this regimen and continued with routine surveillance MRI scans (FIG. 3) and medical follow up. The subject continued administration of the biomass composition, and has had progression free survival for over three years and five months following the initial diagnosis. Collectively, these results demonstrated that treatment using the biomass compositions disclosed herein after concurrent chemoradiation therapy treatment resulted in a decrease in tumor size, and an increase in progression free survival.

### Example 4: Human Study

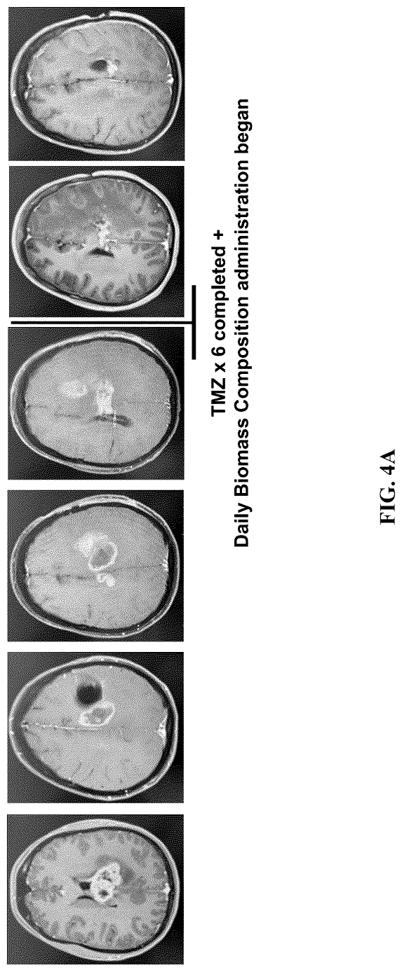

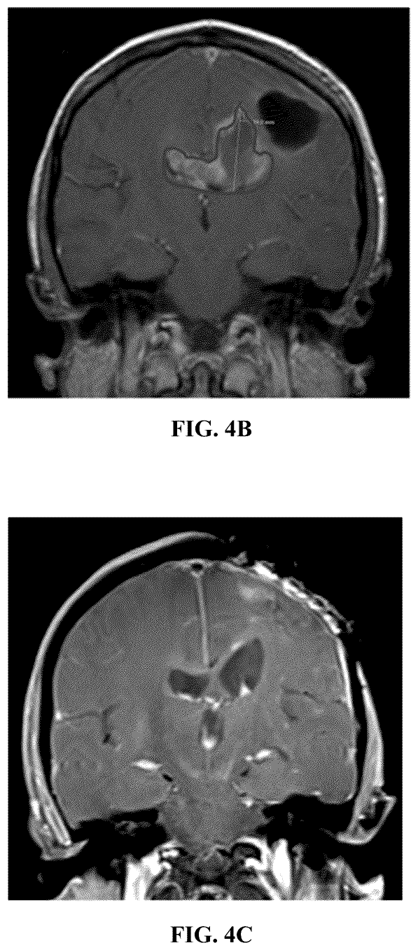

A 41-year-old female subject presented with headaches and gait instability. An MRI of the subject's brain with gadolinium revealed a 3.5×3.0 cm left frontal, intraventricular mass crossing the corpus callosum (FIG. 4A and FIG. 4B). The mass was resected, and a biopsy was performed. The biopsy confirmed a GBM diagnosis that was IDH1 wild-type and the MGMT promotor was methylated. The subject then went on to complete six weeks of concurrent TMZ with radiotherapy. Early on in the subject's chemoradiotherapy course, the subject was administered one dose of bevacizumab. Unfortunately, the subject's course was complicated by wound dehiscence soon after the completion of chemoradiotherapy. The subject underwent a repeat craniotomy with washout of the abscess followed by one month of intravenous antibiotics. Subsequently, the subject began the first cycle of maintenance temozolomide. One month later, the subject's infection recured and the subject was place on antibiotics again. After another month of antibiotics, the subject started the second cycle of maintenance TMZ. The subject went on to complete six total cycles of maintenance TMZ without further infectious complications.

Soon after completing the sixth cycle, the subject was administered formulation A described in Example 2, at 40 mL per dose, twice daily. Approximately 2 months later, a new MRI was performed which revealed significant involution of the contrast enhancing portion of her tumor centered in the posterior corpus callosum, as well as new left hemispheric cerebral edema. Notably, records indicated that the subject had difficulty fully tapering off the antiepileptic medications, which corresponds to the point at which the MRI images initially demonstrated findings consistent with cerebral edema. After 3 months of daily administration of formulation A, the subject's tumor was reduced by 30% and the midline shifting back to center. Approximately 6 months after initiation of treatment using the biomass composition according to formulation A, the subject's tumor shrunk by 75%. However, eight months later, during which the subject was administered the biomass composition twice daily, another MRI revealed further regression of the tumor with resolution of the left hemispheric edema. After 11 months of twice daily administration of formulation A, the subject was tumor free (FIG. 4C). As shown in FIG. 4C, there was complete resolution of enhancing disease in the brain with no evidence of glioblastoma. The butterfly-shaped space observed in the image was the site where the original mass was located (FIG. 4B). The subject has continued administration of the biomass composition, and had progression free survival for five years and three months since the initial diagnosis. Collectively, these results demonstrated that treatment using the biomass compositions disclosed herein after six cycles of TMZ treatment resulted in a decrease in tumor size, and an increase in progression free survival.

### Example 5: Human Study

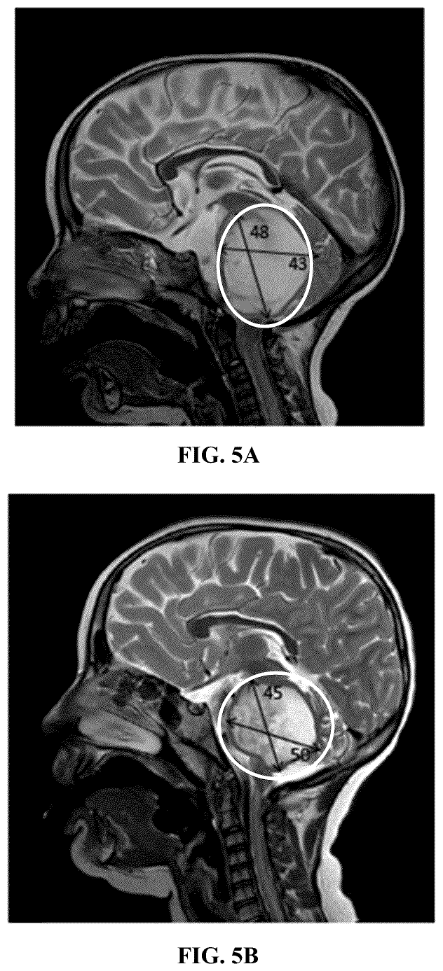

A 9-year-old female subject was diagnosed with diffuse intrinsic pontine glioma (DIPG) with a tumor of 48×43 cm (FIG. 5A). Since the current standard of care for DIPG is palliative the subject did not receive chemotherapy or radiation therapy. Instead, the subject enrolled in the clinical trials disclosed herein and was administered formulation A described and prepared according to Example 2. After two years of twice daily administration, the subject's tumor shrunk by 15% (FIG. 5B), and their vision, which was partially lost, returned. After 5 years of continued administration, the subject's tumor was reduced by 50%. Collectively, these results demonstrated that a monotherapeutic treatment using the biomass compositions disclosed herein resulted in a decrease in tumor size, and an increase in progression free survival.

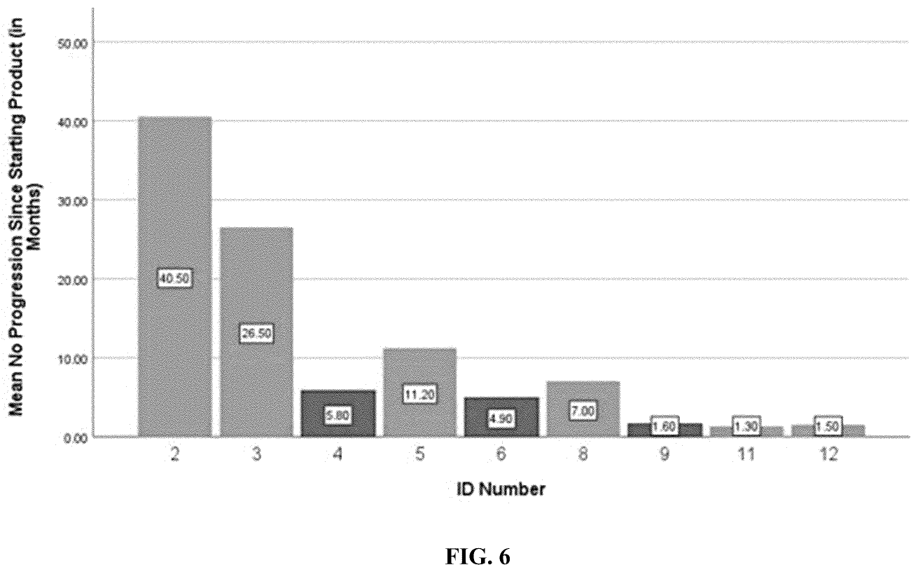

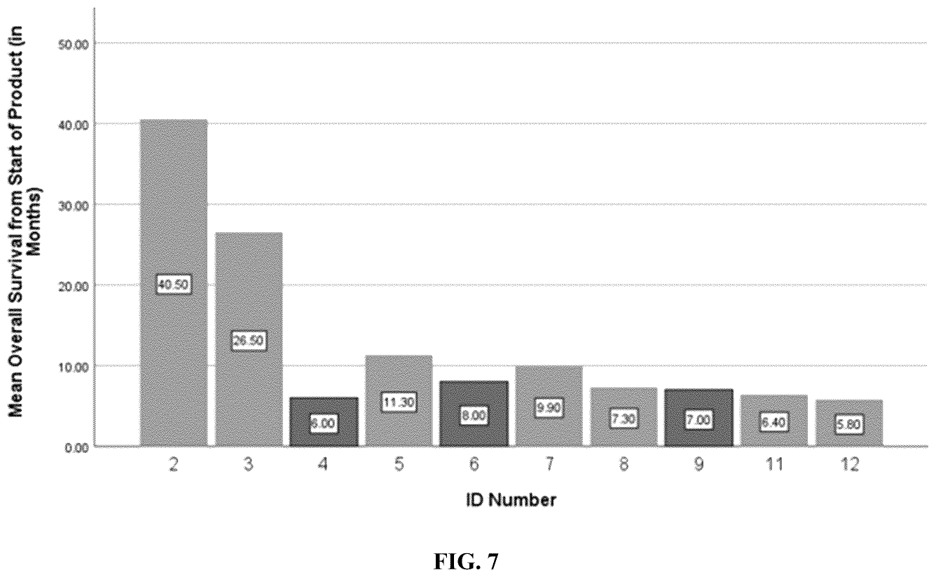

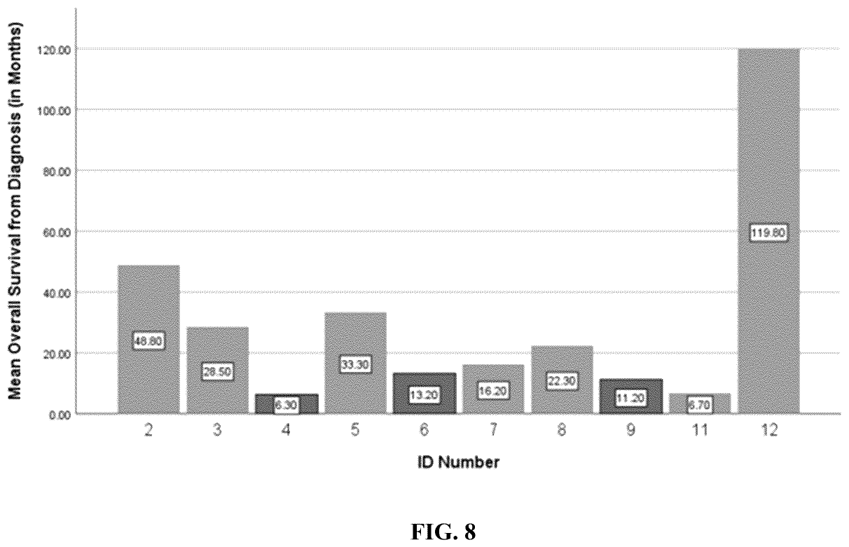

As described above, the biomass compositions disclosed herein demonstrated a decease in tumor volume/size and an increase in progressional free survival (PFS) in subjects. FIG. 6 depicts the progression free Survival (PFS) represents number of months with no tumor growth or with tumor shrinkage after administration of formulation A began. The median PFS is 5.8 months, with a minimum of 1.3 months and an ongoing maximum of 40.5 months (FIG. 6). The median overall survival from since starting the clinical trial is 7.6 months with a minimum of 5.8 months and an ongoing maximum of 40.5 months (FIG. 7). The median overall survival from diagnosis is 19.25 months as compared to 17 months with just the current standard of care (FIG. 8). There was a minimum of 6.3 months and an ongoing maximum of 120 months. Notably, there are 6 subjects in the ongoing clinical trial with excellent survivability of 120, 49, 33, 28, 22, and 16 months since diagnosis (FIG. 8). Without wishing to be bound by theory, multiple mechanisms are considered underlying these effects, with the common theme of modifying the tumor microenvironment at the cellular and molecular levels. The biomass composition is hypothesized to induce immunogenicity and mitigate tumor invasiveness.

### Example 6: Effects Filtered and Unfiltered Biomass Compositions on Glioblastoma Cell Lines

This example describes the anticancer effects of the filtered and unfiltered biomass compositions on human glioblastoma cell lines. Approximately 56 packets each containing 20 mL of formulation A described in Example 2, were thawed and diluted with phosphate-buffered saline. Upon solubilization of formulation A, some insoluble precipitate was observed. An aliquot of the solution was then filtered with either a 70 uM pore filter or a 0.45 uM pore filter. Solutions filtered with the 70 uM filter still demonstrated evidence of insoluble precipitate, however, solutions filtered with the 0.45 uM pore filter were fully soluble.

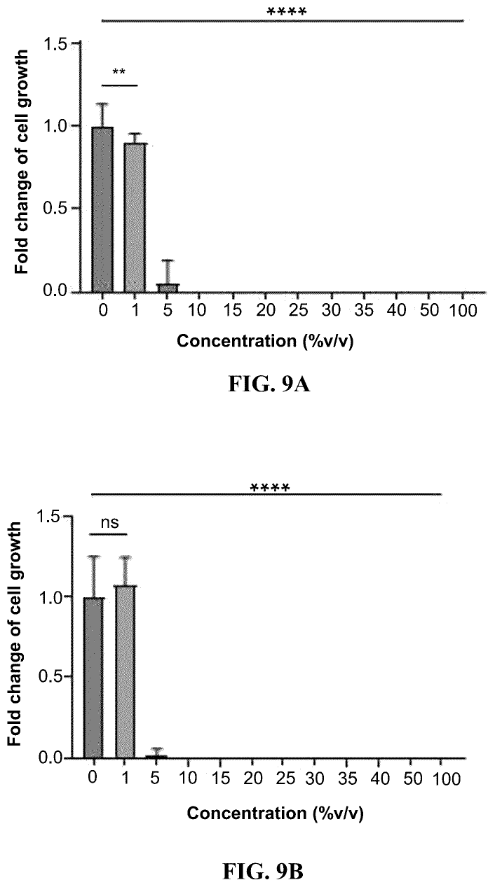

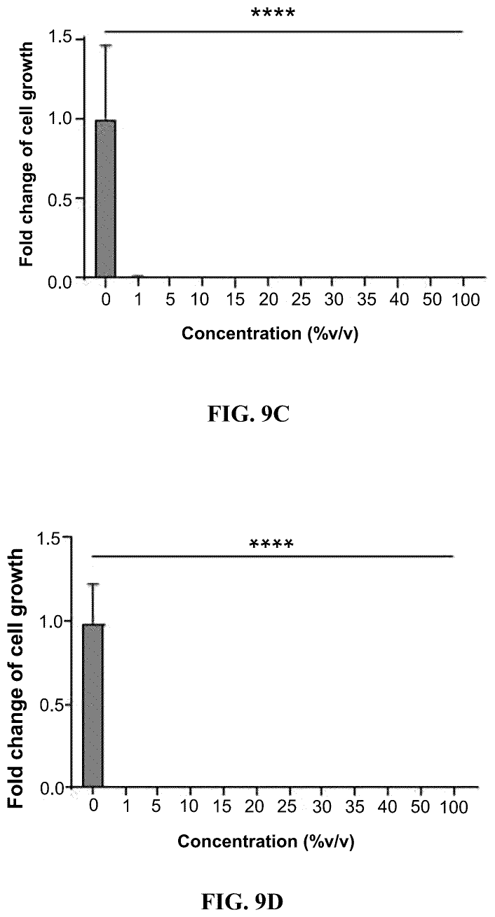

Next, the anti-tumor activity of the biomass compositions was assessed in two representative glioblastoma cell lines in in vitro cell culture using a cell viability assay. Briefly, for each cell line, cells from were treated with increasing concentration of the biomass compositions disclosed herein, and the fold change of cell growth was then assessed. The first cell line was an epithelial-like cell line that was isolated from the right frontal parieto-occipital cortex of a glioblastoma patient (LN229 cells). The second cell line was a primary human glioblastoma cell line (GBM-8). A wide range of concentrations of the biomass compositions were diluted in standard media, ranging from 0% (untreated) to 100% by volume of the biomass composition. As shown in FIGS. 9A-9B, LN229 cells were sensitive to the biomass compositions disclosed herein with nearly 100% tumor kill observed in the presence of 5% v/v filtered or unfiltered biomass composition, as quantified by live tumor bioluminescence. Notably, the filtered biomass composition demonstrated similar tumor killing activity as compared to the unfiltered biomass composition. This data indicates the active biomass components are fully soluble in water. GBM8 cells were even more sensitive to the biomass compositions disclosed herein with nearly 100% tumor kill observed in the presence of 1% v/v filtered or unfiltered biomass composition (FIGS. 9C-9D).

### Example 7: Off-Target Toxicity of the Biomass Compositions on Normal Brain Tissue

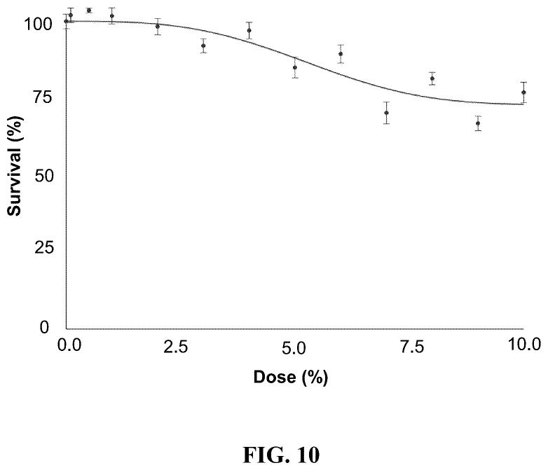

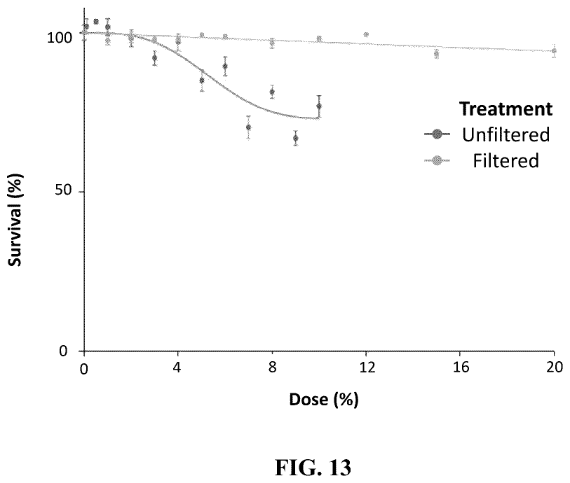

This example describes the off-target toxicity of the biomass compositions to normal brain tissue. For this assay, living organotypic brain slice cultures (OBSCs) derived from rat pup brains were used. Minimal toxicity from the biomass compositions on OBSCs was observed at concentrations between 0.1% and 4% v/v of the unfiltered biomass compositions (FIG. 10). The low brain tissue toxicity observed was in the range where high tumor kill was observed. This data indicates anti-tumor selectivity. At higher concentrations, such as between 5-10% of the biomass compositions, increased toxicity was observed but there was still 75% survival. 0.45 uM filtered biomass composition displayed less off-target toxicity to living organotypic brain slice cultures than unfiltered biomass composition (FIG. 13).

### Example 8: Effects Filtered and Unfiltered Biomass Compositions on Glioblastoma Cell Lines

This example describes the dose-dependent anticancer effects of the filtered and unfiltered biomass compositions on representative glioblastoma cell lines. The aliquot of the solution filtered with the 0.45 uM pore filter from Example 6 was used as the ‘filtered’ sample in this experiment. The first cell line was a LN229 cell line. LN229 cells were treated with varying concentrations of the filtered biomass composition ranging from 0% v/v to 5% v/v concentration (FIGS. 11A-11B). Cells treated with the unfiltered biomass composition demonstrated a dose-dependent decrease in cell growth (FIG. 11B). However, as shown in FIGS. 11A, the filtered biomass composition was four to five times less potent than the unfiltered biomass composition.

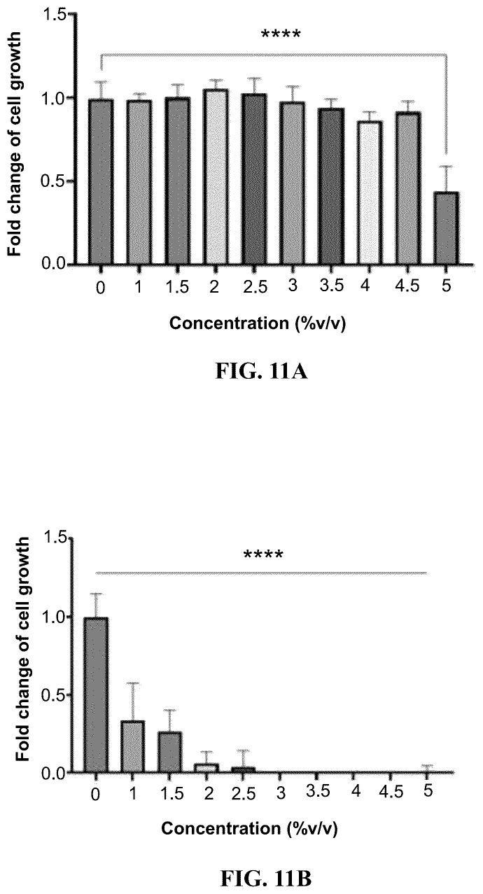

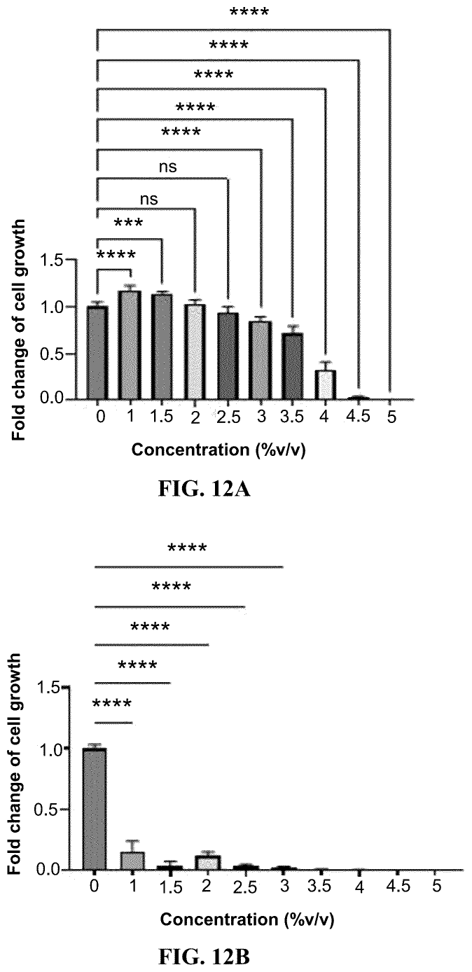

The second cell line was U87, a cell line with epithelial morphology that was isolated from malignant gliomas. U87 cells were treated with varying concentrations of the filtered biomass composition ranging from 0% v/v to 5% v/v concentration (FIGS. 12A-12B). U87 cells treated with the unfiltered biomass composition demonstrated a decrease in cell growth (FIG. 12B). However, as shown in FIGS. 12A, the filtered biomass composition was four to five times less potent than the unfiltered biomass composition, but a dose-dependent decrease in cell growth was observed.

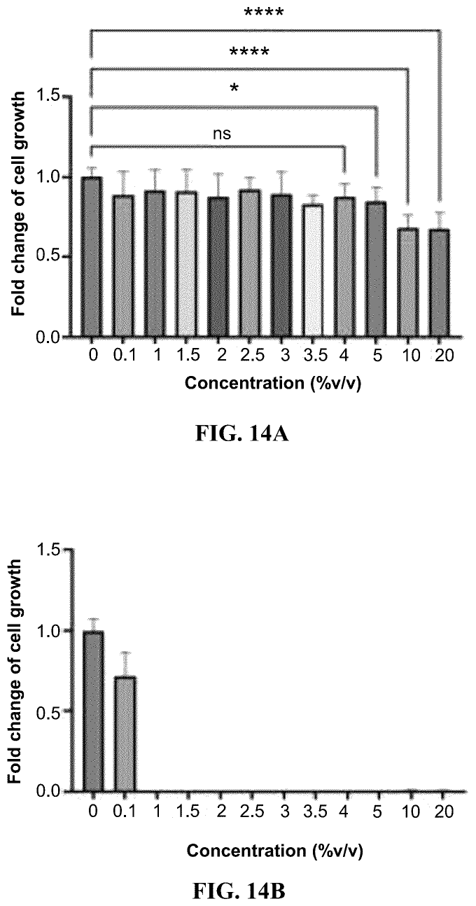

For the third cell line, GBM-8, a different aliquot filtered with a 0.22 uM pore filter was used to assess the activity of the filtered biomass compositions disclosed herein. The biomass composition that was filtered with a smaller 0.22 uM pore filter displayed minimal activity as evidenced by the modest change in cell growth even at high concentrations of 20% (FIGS. 14A-14B). This modest change was compared to the cell growth of GBM-8 cells treated with biomass compositions filtered with a 0.45 uM pore filter. As described in Example 6, GBM-8 cells were potently killed at low doses of the 0.45 uM filtered biomass composition (FIGS. 9C-9D). Collectively, these results demonstrated that the biomass compositions disclosed herein contains one or more active agent(s) that were able to pass through a 0.45 uM pore filter, but were unable to pass through a 0.22 uM pore filter.

### Example 9: Effects Filtered and Unfiltered Biomass Compositions on Glioblastoma Cell Lines Grafted on Organotypic Brain Slice Cultures (OBSCs)

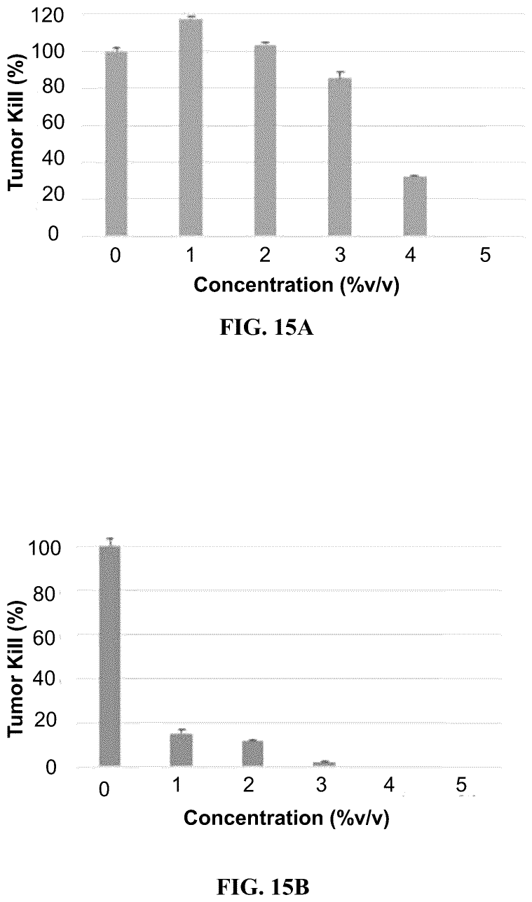

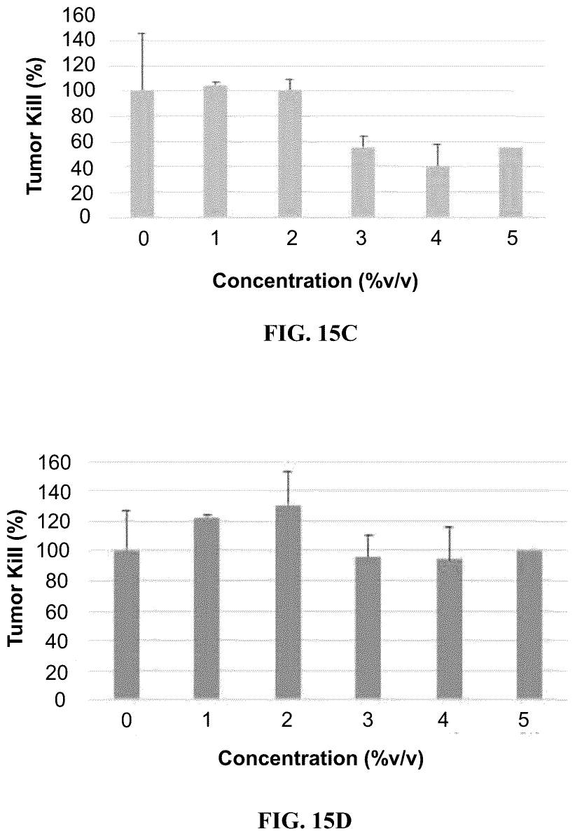

This example describes the ability of the biomass compositions to kill U87 glioblastoma cells grafted on an organotypic brain slice cultures (OBSC) platform. The U87 cells were grafted atop the OBSC which was in direct contact with the media mixed with varying concentrations of the biomass compositions. For the biomass composition to kill the cancer cells in this platform, the active agents would have to be small or soluble enough to diffuse through the OBSC tissue and reach the U87 cells. In this experiment, the filtered and unfiltered biomass compositions exhibited decreased potency against OBSC-engrafted U87 cells (FIGS. 15C-15D), as compared to the same U87 cells grown in a plastic dish where the biomass composition directly contacted the tumor cells. This data indicates that a majority of the biomass composition was settled at the bottom of the OBSC platform, and was unable to diffuse through the OBSC.

### Example 10: Effects Filtered and Unfiltered Biomass Compositions on Mouse Glioblastoma Cell Lines

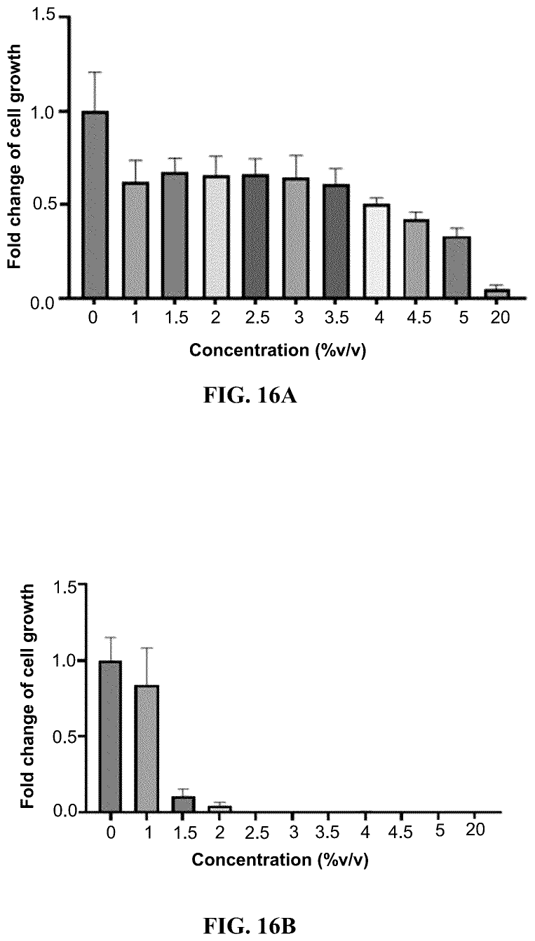

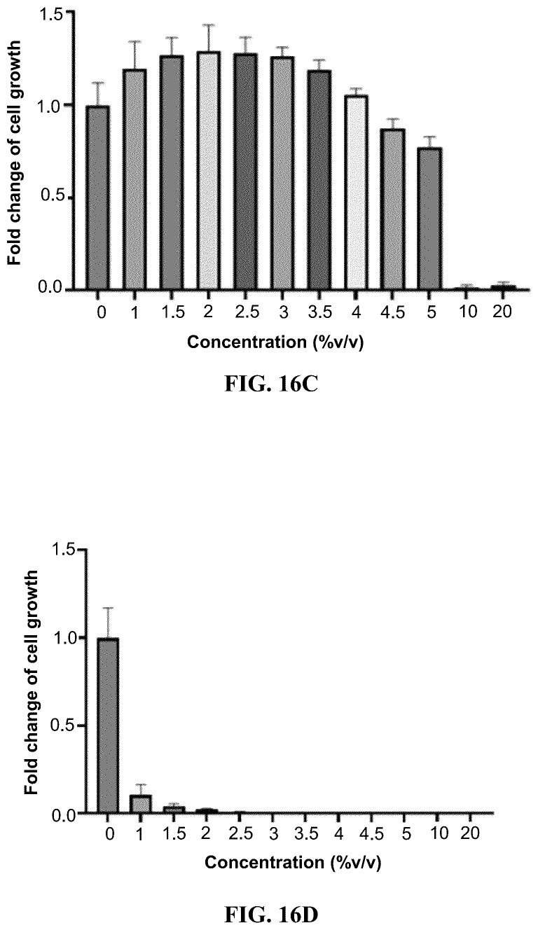

This example describes the anticancer effects of the filtered and unfiltered biomass compositions on mouse glioblastoma cell lines in in vitro cell culture. The first cell line was KR158B which exhibited many features similar to human GBM, including invasive growth, rapid proliferation, and the ability to form tumor spheres. The second cell line was GL261 which mimicked the aggressive and invasive characteristics of human GBM. A wide range of concentrations of the filtered and unfiltered biomass compositions were diluted in standard media, ranging from 0% (untreated) to 20% by volume of the biomass composition for each cell line. As shown in FIG. 16B, the unfiltered biomass composition exhibited high potency against KR158B at a low concentration of 1.5% v/v, as compared to the 0.45 uM filtered biomass composition which demonstrated less potency but still induced cell death at 20% v/v (FIG. 16A). As shown in FIG. 16D, the unfiltered biomass composition exhibited high potency against GL261 cells at a low concentration of 1% v/v, as compared to the 0.45 uM filtered biomass composition which demonstrated less potency but still induced cell death at 10% v/v (FIG. 16C). Interestingly, low concentrations of the filtered biomass composition induced ˜40% tumor kill in KR158B cells, this indicated the presence of potent active compounds in the filtered solution.

### Example 11: In Vitro Efficacy of the Biomass Compositions Following Centrifugation Against LN229 and GL261

This example describes the potential correlation between the various centrifuged fractions of biomass composition obtained at different speeds and its antitumor activity against a human glioblastoma cell line (LN229) and a mouse glioblastoma cell line (GL261). The unfiltered biomass composition contained both large and small debris which imparted significant heterogeneity into the complex mixture. The cell lines LN229 and GL261 were labeled with optical reporter genes for fluorescence (mCherry) and bioluminescence (Firefly luciferase) quantification of tumor growth. The culture media used for both cell lines was Dulbecco's Modified Eagle Medium (DMEM)+10% Fetal Bovine Serum (FBS)+1% Penicillin-Streptomycin (P/S). The cell lines were cultured in T-175 flasks kept in the incubator in optimal conditions of temperature (37° C., CO2 conc. (5%) and humidity (90%)). Visual confirmation of cell growth and cell confluency of >70% in the culture flask were made and the cells were transferred to the experimental plates (96 wells) in the following steps: on day 0, cells were seeded in a 96-well plate, and each well seeded with 10,000 tumor cells. On day 1, the cells were treated with increasing concentrations of the biomass composition corresponding to each experimental group. On day 4, live tumor bioluminescence (BLI) readings were performed.

The experimental groups were: (1) biomass composition (no centrifugation); (2) biomass composition supernatant after settling for 2 hours; (3) biomass composition supernatant after 70 rcf for 5 min at room temp; (4) biomass composition supernatant after 277 rcf for 5 min at room temp; (5) biomass composition supernatant after 627 rcf for 5 min at room temp; (6) biomass composition supernatant after 1109 ref for 5 min at room temp; (7) biomass composition supernatant after 1708 ref for 5 min at room temp; (8) biomass composition supernatant after 2509 ref for 5 min at room temp; (9) biomass composition supernatant after 10310 ref for 5 min at room temp; and (10) biomass composition supernatant after 14580 ref for 5 min at room temp (No centrifuge, 2 hour settled (but not centrifuged), 70 ref, 277 ref, 627 ref, 1109 ref, 1708 ref, 2509 ref, 10310 ref, 14580 ref). The doses of the biomass composition used in these experiments were based on volume-to-volume (V/V) ratios of the biomass composition and culture media, comprising 12 experimental groups for both glioblastoma cell lines, as follows: 0.0, 0.5, 1.0, 1.5, 2.0, 2.5, 3.0, 3.5, 4.0, 4.5, 5.0 and 5.5% v/v.

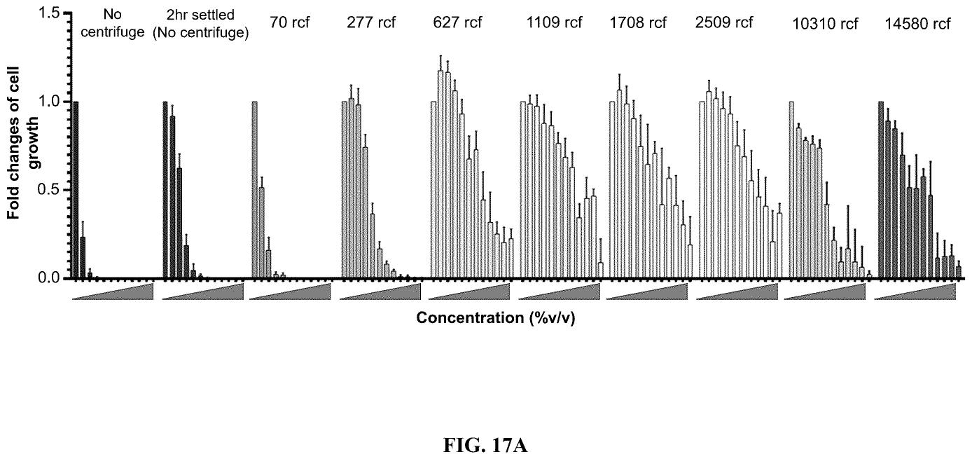

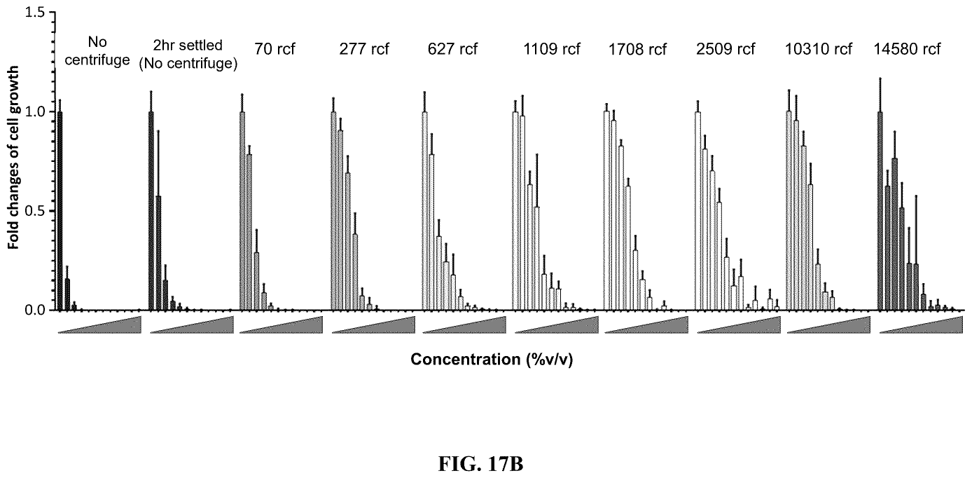

A potent response to the biomass composition treatment in both cell lines across all experimental groups was observed, with GL261 showing greater sensitivity to the extract. In both cases (FIGS. 17A-17B), as the centrifugation speed increased, the antitumor potency progressively decreased. A large decrease in potency occurred between 70 rcf and 277 rcf, and again when centrifugation speed was increased to 627 rcf. Above 627 rcf, there were minimal additional decreases in potency, indicating a majority of the remaining active molecules were soluble in the solution. This data indicates that soluble biomass composition after high-speed centrifugation is amenable to intravenous administration, which greatly increases bioavailability and ability to reach the tumor site.

### Example 12: In Vitro Efficacy of the Unfiltered Biomass Compositions Following Centrifugation Against GL261

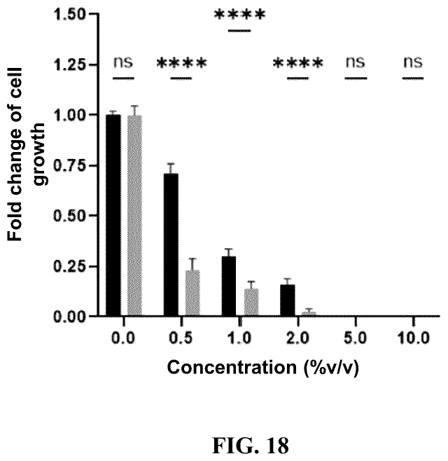

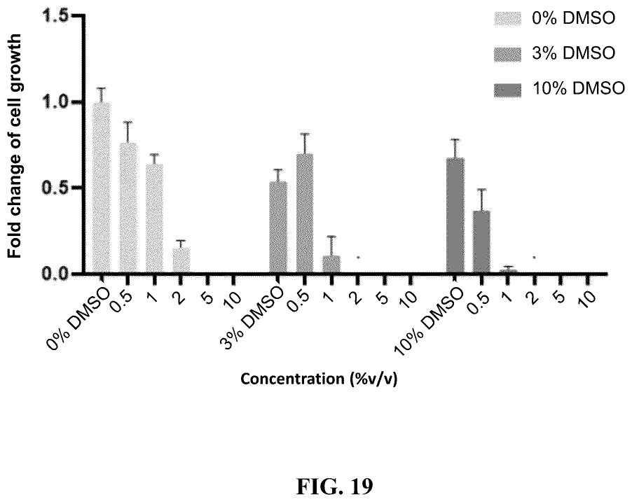

This example was used to determine whether any portions of these debris that remained after the biomass composition settled contributed to therapeutic effect against cancer. Upon imaging under a microscope, the debris looked like cellular debris and small chunks of tissue, therefore the supernatant of the biomass composition was added to GL261 cancer cells (FIG. 18). Indeed, the centrifuged the biomass composition supernatant was not as potent as uncentrifuged the biomass composition, indicating that components in these large chunks and cellular debris had therapeutic effect (FIG. 18). Alongside the data described in Examples 8-11 which compared filtered and unfiltered the biomass composition, these results indicated that there are distinct levels of bioactive molecules in the filtered the biomass composition component, the unfiltered the biomass composition with large tissue chunks removed, and the large tissue chunks themselves (which may slowly dissolve in culture over time). Since large tissue chunks and other debris cannot be run through a column for characterization, all potentially bioactive molecules must therefore be dissolved in solution. Small amounts of DMSO (0%, 3% and 10%) were added to the biomass composition product. As shown in FIG. 19, adding increasing amounts of DMSO to the biomass composition increased killing of KR158B cells in vitro (FIG. 19). This finding indicated that DMSO or other solubilizing agents may increase therapeutic potency and bioavailability of the biomass composition by solubilizing more bioactive compounds.

Examples 6-11 illustrated that the biomass composition was a potent anti-tumor compound against all human and murine GBM cell lines tested in vitro thus far with very little off-target toxicity in the therapeutically relevant dose range.

### Example 13: In Vivo Toxicity Studies

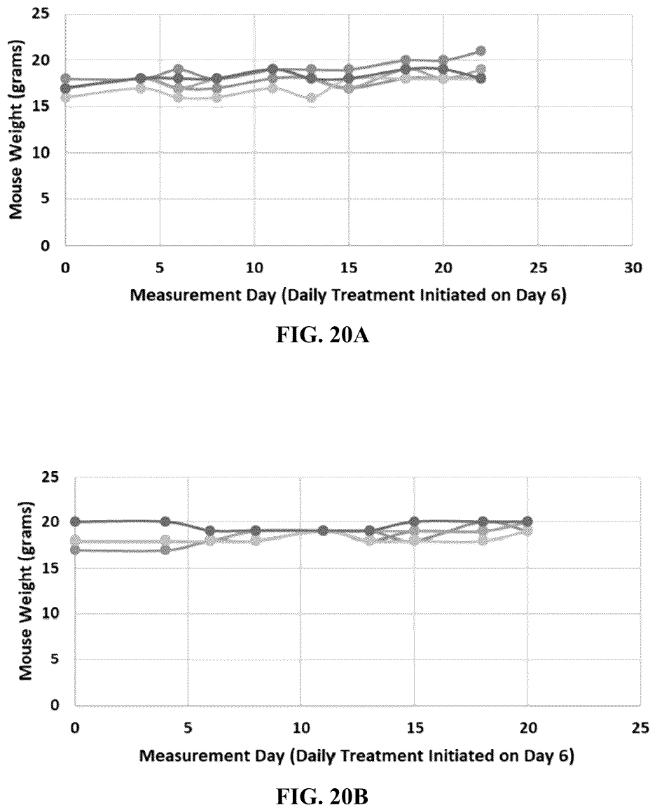

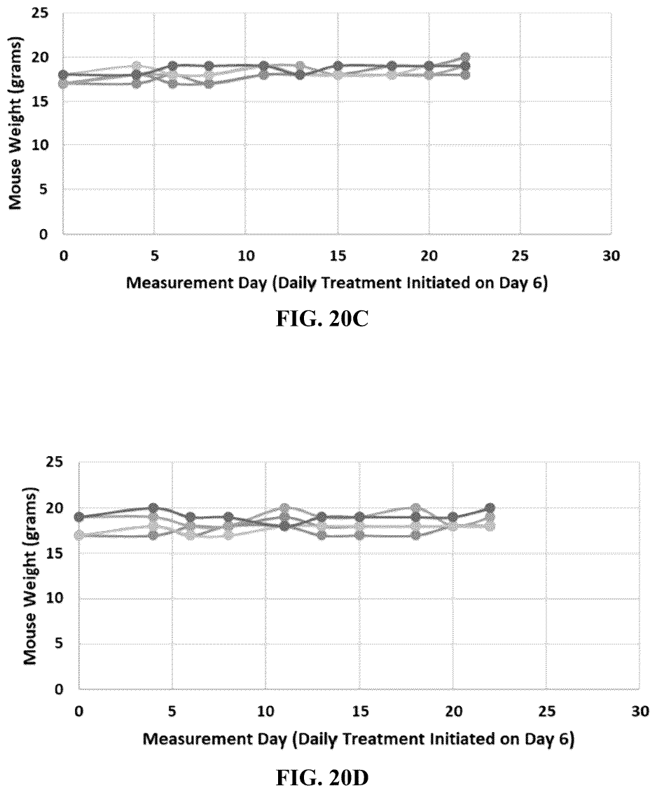

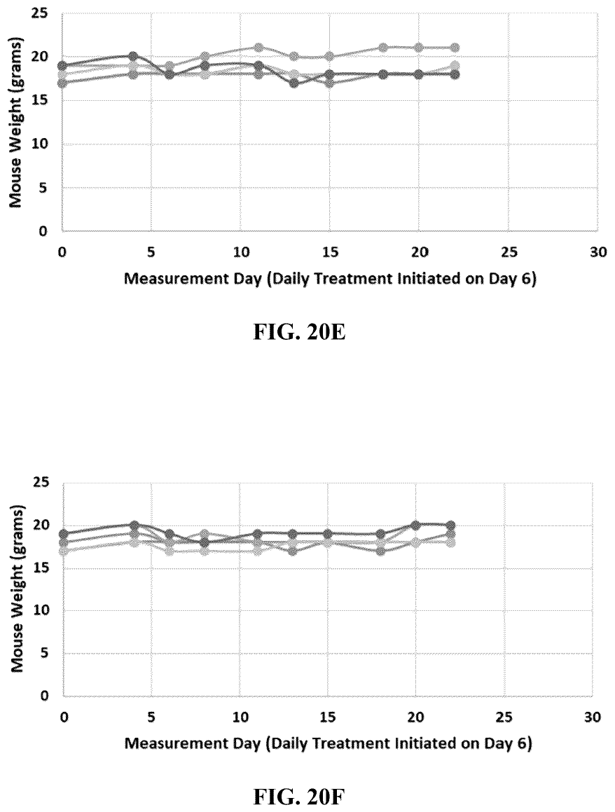

This example was used to determine if there are any negative effects of daily administration of the biomass composition described herein in live mice. Two different strains of mice were used, the first was a severely immunocompromised strain called NSG; and the second was an immunocompetent strain called C57Bl/6. Mice were administered by oral delivery: a low human-equivalent daily dose (0.57 ml/kg of body weight, which equals 40 ml per day for a 70 kg human), or a high human-equivalent daily dose (5 ml/kg of body weight, which equals 350 ml per day for a 70 kg human) of the biomass composition (BMC) as compared to a negative saline control. Five mice were used for each experimental condition, and the body weights of each animal was measured every other day. Plots of mouse body weights, divided by treatment group (n=5 mice per group) are shown in FIGS. 20A-20F. No significant changes in body weight was observed in any of the mice treated with low dose, high dose or saline control. Overall, this data indicates that both the low and high dose of the biomass compositions described herein were well tolerated in both immunocompromised and immunocompetent mice. Body Condition Scores for all mice at all time points remained at 3 for the duration of the study (data not shown).

The differences in blood chemistry in treated and untreated mice, including features and numbers of red and white blood cells, platelets, biomarkers of liver function and biomarkers of kidney function were also assessed. Mice were administered by oral delivery: a low dose suspension (1.14 ml/kg) of the biomass composition which were prepared before each administration, or a high dose suspension (5 ml/kg) of the biomass composition. Phosphate-buffered saline was used to dilute the biomass composition for the low dose treatments and also used as a negative control group. Five mice were used for each experimental condition. The biomass compositions were administrated by oral gavage as a single daily dose for 28 days using a maximum volume per mouse of 100 uL. Blood collection was performed as a terminal procedure at Day 28 before the whole-body perfusion took place. Approximately, ⅓ of the amount of blood collected from each animal was treated as whole blood and put in tubes for the complete blood count (CBC) test, ⅓ was processed to plasma by using heparinized tubes and centrifugation for proteomic analysis, and the last ⅓ was processed to serum in separator tubes for basic organ functional test (liver/kidneys).

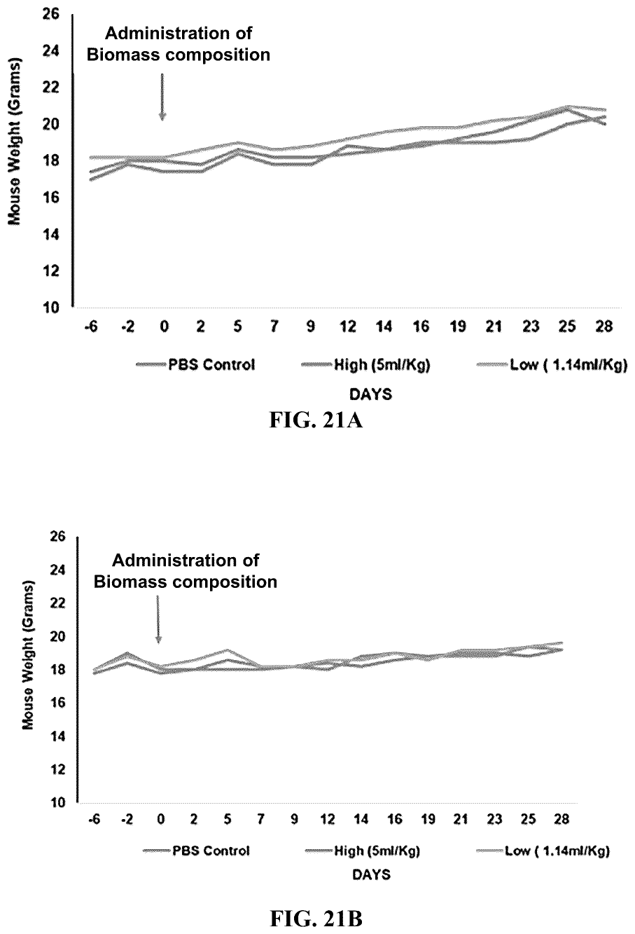

Weight loss was considered an important indicator of toxicity in mice. FIGS. 21A-21B demonstrated the changes in body weight of the mice throughout the study. No significant weight loss was ever observed, particularly during the oral administration of the biomass composition. Small fluctuations in body weight, typically within a ±5% range, are considered normal. A 20% weight loss or the onset of severe pain was established as a humane endpoint in mice in this study, at which point the animal would be removed from the study or euthanized due to a significant decline in health. These events were not observed in any of the treatment groups for both mouse strains.

The Complete Blood Count (CBC) performed on the final day of the biomass composition administration indicated that all parameters appear to be within normal reference ranges, with no significant deviations observed. Each mouse's hemoglobin, hematocrit, red blood cells, white blood cells, platelets, and reticulocytes are all within expected limits, indicating a healthy hematologic profile. While slight differences were noted when compared to internal controls, in the case of reticulocytes from the immunocompromised mice and hematocrits, mean corpuscular hemoglobin concentration (MCHC) and mean corpuscular volume (MCV) from the immunocompetent mice, these variations are minimal and unlikely to have any meaningful impact on normal hematologic function. As such, they are not considered physiologically significant.

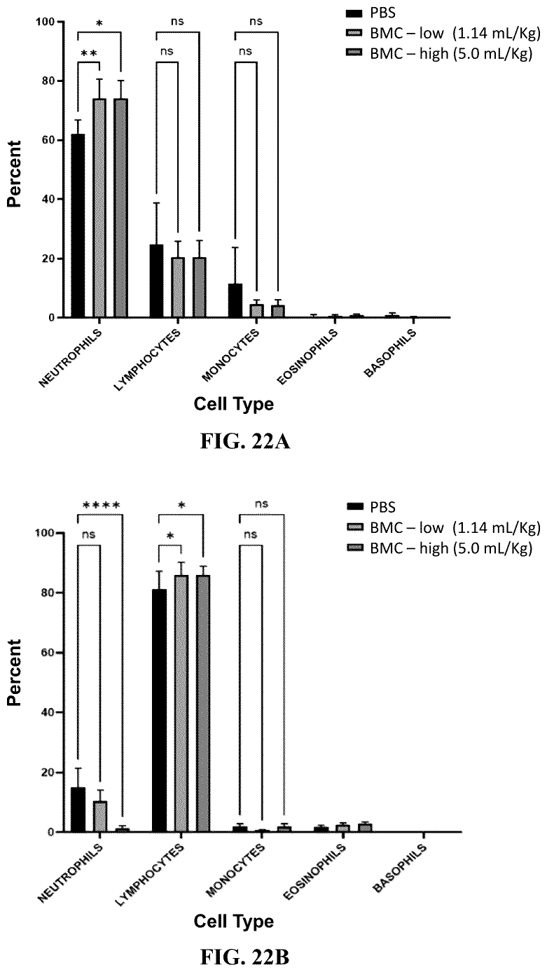

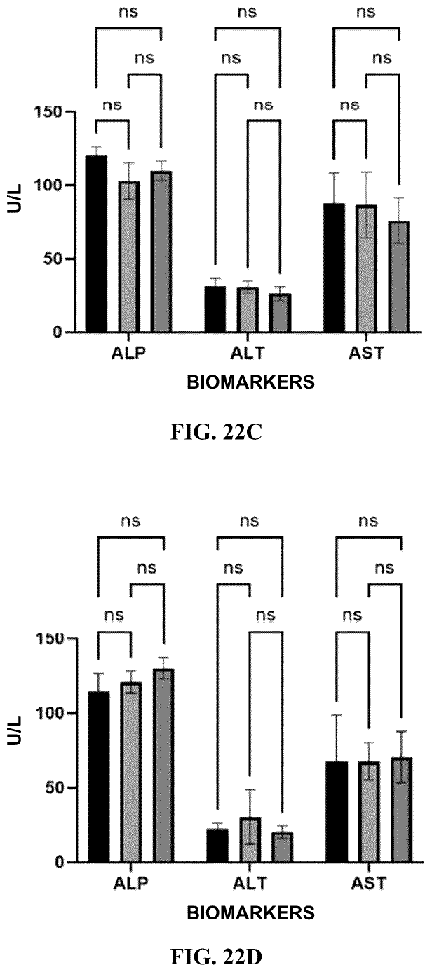

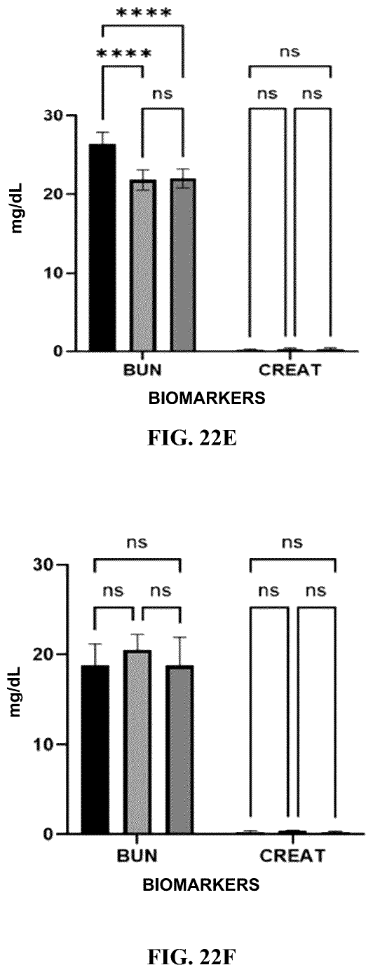

Interestingly, the individual white blood cell counts (neutrophils, lymphocytes, monocytes, basophils, and eosinophils) for each mouse strain (FIGS. 22A-22B) showed distinct profiles, particularly for neutrophils and lymphocytes. This was expected, given that we are working with both an immunocompromised strain and an immunocompetent strain. The immunocompromised mice are known to lack functional T cells, B cells, and NK cells, or have these cells in extremely low numbers. In FIG. 22A, immunocompromised mice exhibited upregulated neutrophil levels, likely as a compensatory response to the impaired function of other immune cells, such as lymphocytes. In contrast, lymphocyte counts in immunocompetent mice appear to be within the normal range for an immunocompetent strain (FIG. 22B). A significant decrease in neutrophil count was observed in the high dose group of immunocompetent mice, compared to the control group, as illustrated in FIG. 22B. The underlying cause of this response was unclear, and therefore further histopathological analysis of the collected tissues in this study was required to assess potential increases in neutrophil clearance, enhanced tissue sequestration, or a reduction in neutrophil production.

As part of the clinical chemistry analysis, basic functional tests for liver and kidney function were conducted across all treatment groups for both mouse strains. ALT (Alanine Aminotransferase), ALP (Alkaline Phosphatase), and AST (Aspartate Aminotransferase) are biomarkers present in the liver but also in various tissues, including heart, bone, and muscles. While these enzymes were primarily used to assess liver function, they can also provide valuable insights into conditions affecting other organs. No significant negative changes in the levels of these enzymes were observed in any of the mouse strains or experimental groups, indicating normal liver function across the board (FIGS. 22C-22D).

Blood Urea Nitrogen (BUN) and creatinine (CREAT) were key biomarkers used to assess kidney function. These markers, measured through blood tests, offer valuable insights into the kidneys' ability to filter waste products from the blood. As shown in FIGS. 22E-22F, a decrease in BUN levels was observed in the treatment groups of immunocompromised mice, while no significant change was noted in the immunocompetent mice. This response was potentially caused by several factors, including factors related to kidney function, hydration status, or changes in protein metabolism; however, it was also possible that the observed difference was not physiologically significance as the BUN values fall within the normal range for the immunocompromised mice. To draw a reliable conclusion of this change, it was necessary to analyze the histopathological findings on the tissues collected to confirm or correlate any potential kidney impairment. Overall, these results demonstrate that the administering of low or high dose of biomass composition as disclosed herein did not result in increased toxicity in either immunocompromised or immunocompetent mice.

### Example 14: Effects Biomass Compositions on Glioblastoma Cell Lines

This example describes the anticancer effects of the filtered and unfiltered biomass compositions on glioblastoma cell lines. Approximately 56 packets each containing 20 mL of formulation A described in Example 2, were thawed and diluted with phosphate-buffered saline. Five different types of brain cancer cell lines (LN229, GL261, U87, KR158, and GBM8 cells) were each treated with a range of concentrations of the biomass composition diluted in standard media (0.1%, 1%, 2%, or 3% v/v) as compared to an untreated control (0% v/v). The percent survival of the five different types of brain cancer cell lines was quantified 72 hours after treatment.

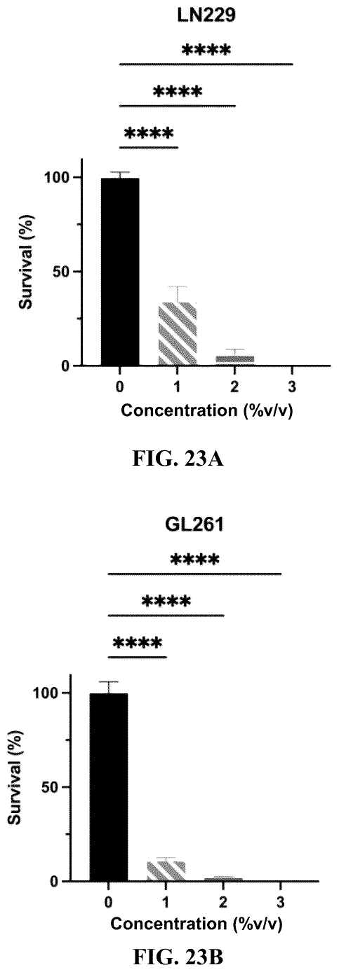

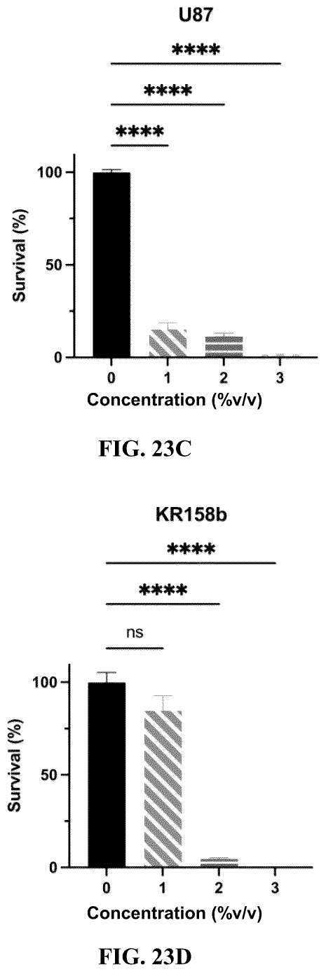

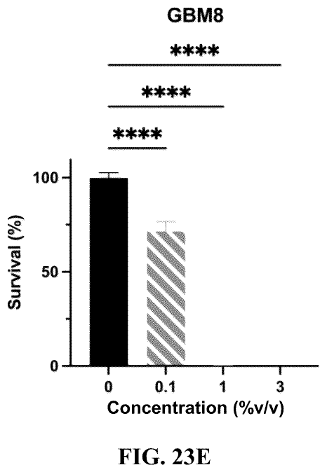

As shown in FIGS. 23A-23C, LN229, GL261, and U87 cells were sensitive to treatment of 1% v/v in media of the biomass composition as the percent survival reduced below 50%. The KR158b cell line was less sensitive at 1% v/v of the biomass composition. As shown in FIG. 23D, treatment with 10% v/v of the biomass composition did not result in a significant decrease in percent survival of KR158b cells, however, increasing to 2% v/v resulted in nearly 100% cell death. Interestingly, treatment of GBM8 cells with 0.1% v/v of the biomass composition resulted in a 25% reduction in cell survival, which was further reduced at the 1% concentration. Notably, treatment with 3% v/v of the biomass composition resulted in nearly 100% cell death in all five of the tested cell lines (FIG. 23A-23E). Collectively, this data demonstrates that all five brain cancer cell lines tested were sensitive to treatment with the biomass compositions disclosed herein at low concentrations. Moreover, GBM8 cells were even more sensitive to the biomass compositions disclosed herein with 25% cell death observed in the presence of 0.1% v/v of the biomass composition, and 100% cell death in the presence of 1% v/v of the biomass composition (FIG. 23E).

### Example 15: In Vivo Efficacy of the Biomass Compositions Disclosed Herein in a GL261 Mouse Model

This example was used to determine the in vivo efficacy of the biomass composition described herein in a GL261 mouse model. Two different strains of mice were used, the first was a severely immunocompromised strain called NSG; and the second was an immunocompetent strain called C57Bl/6. Both groups of mice were transduced with G1261 cells, and were simultaneously administered by oral delivery: a low dose of the biomass composition of 1.14 ml/kg (BMC-low), or a high dose of the biomass composition of 5 ml/kg (BMC-high) as compared to a PBS control. Five mice were used for each experimental condition. Tumor growth and body weights of each animal was measured for 15 days.

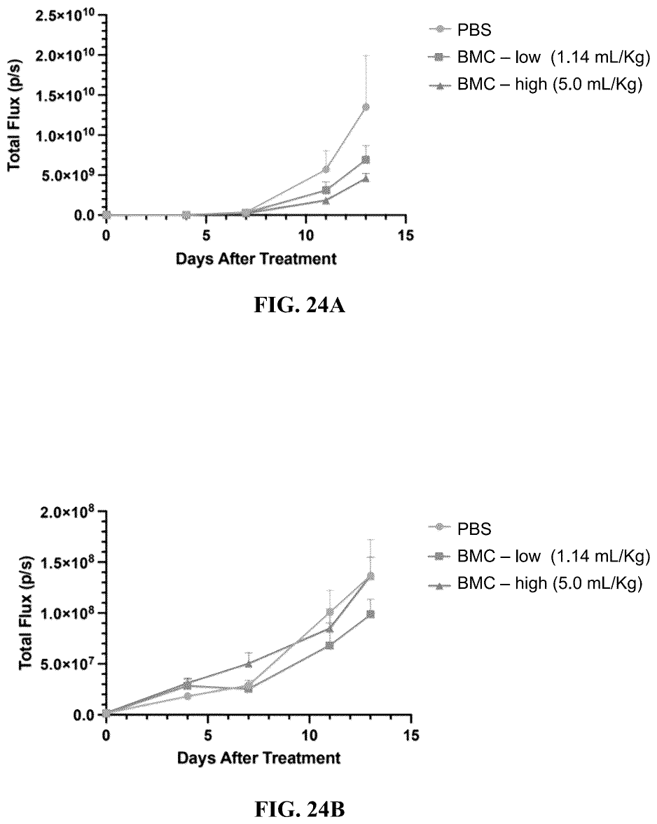

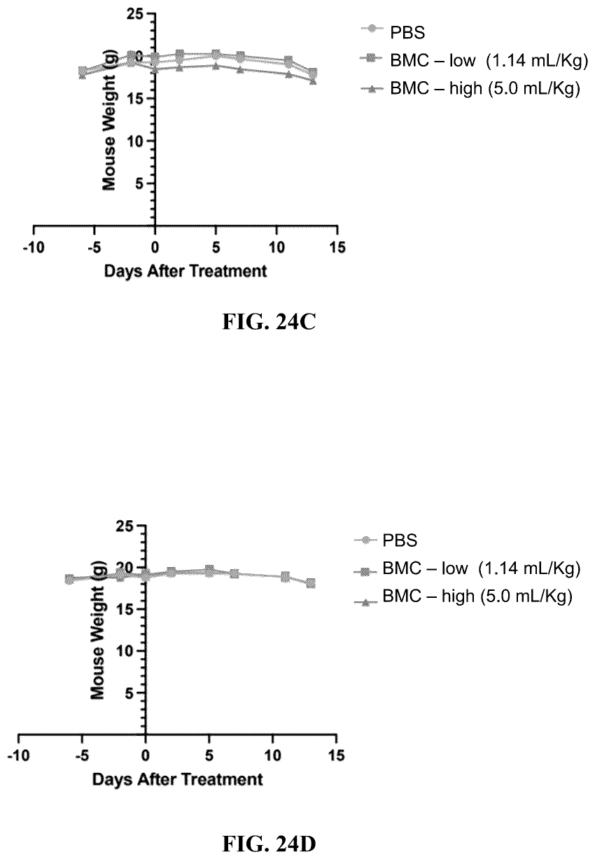

Bioluminescence signals from GL261 tumors implanted in the brains of mice were monitored over time, starting from inoculation (FIGS. 24A-24D). As shown in FIGS. 24A-24B, on the first day of treatment there was no significant tumor reduction between the control and BMC treated groups within each mouse strain, nor were there significant differences in tumor growth following daily oral gavage administration of the biomass compositions at both low and high doses. Notably, the tumor growth in the immunocompromised mice was delayed in both BMC-treated groups as compared to the control group (FIG. 24A). A similar, albeit less pronounced, trend was observed in the immunocompetent mice (FIG. 24B). Thus, both FIG. 24A and FIG. 24B demonstrated a dose-dependent decrease in tumor growth after treatment with the biomass composition. For the immunocompromised mice, according to Tukey's multiple comparison test BMC low dose as compared to PBS has a p-value of 0.4583 while the high-dose has a p-value of 0.2281. For the C57B/L6 mice, this trend was not conserved. As shown in FIG. 24C and FIG. 24D, and consistent with Example 13, there was no significant trends in body weight. Collectively, this example demonstrated that treatment with the biomass compositions disclosed herein resulted in a dose-dependent decrease in tumor growth in immunocompromised and immunocompetent mice.

### Example 16: In Vitro Efficacy of the Biomass Compositions Disclosed Herein in Combination with an Additional Therapy

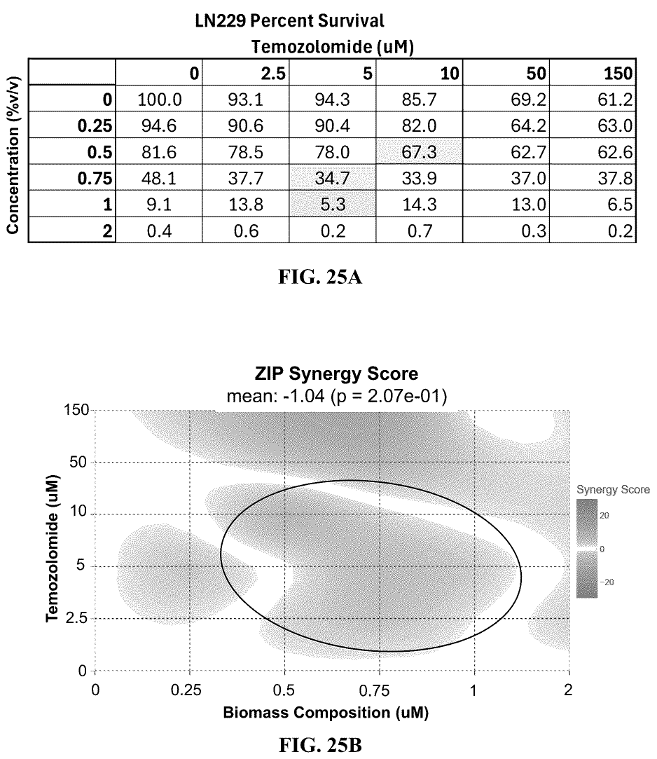

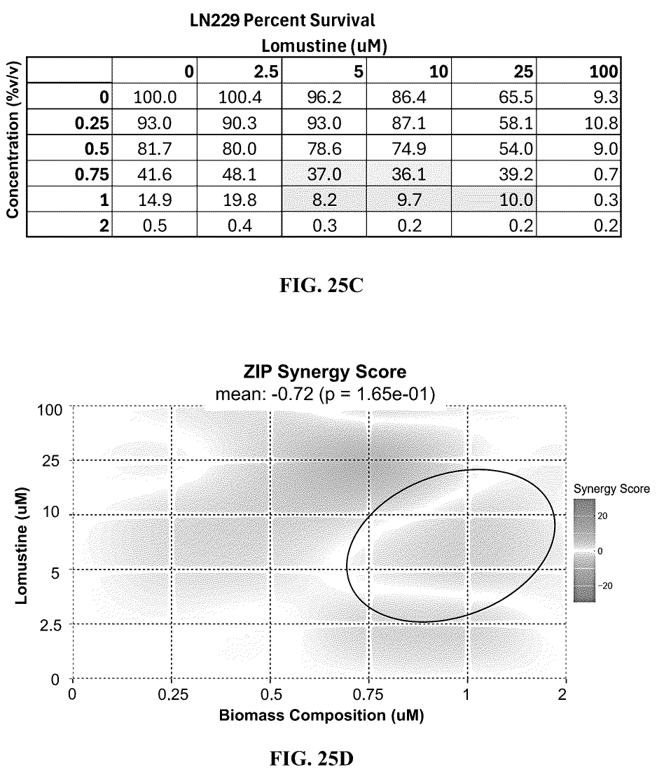

This example was used to determine the in vitro efficacy of the biomass composition described herein in combination with an additional therapy, temozolomide and lomustine, in a LN229 cells. Briefly, the synergistic antitumor activity of the biomass compositions in combination with the additional therapy a human glioblastoma cell line (LN229). The LN229 cells were labeled with optical reporter genes for fluorescence (mCherry) and bioluminescence (Firefly luciferase) quantification of tumor growth. The culture media used for both cell lines was Dulbecco's Modified Eagle Medium (DMEM)+10% Fetal Bovine Serum (FBS)+1% Penicillin-Streptomycin (P/S). The cell lines were cultured in T-175 flasks kept in the incubator in optimal conditions of temperature (37° C., CO2 conc. (5%) and humidity (90%)). Visual confirmation of cell growth and cell confluency of >70% in the culture flask were made and the cells were transferred to the experimental plates (96 wells) in the following steps: on day 0, cells were seeded in a 96-well plate, and each well seeded with 10,000 tumor cells. On day 1, the cells were treated with increasing concentrations of the biomass composition, and increasing concentrations of either temozolomide (μM) and lomustine (μM). The percent survival data from three separate experiments were averaged and the synergistic activity was quantified via ZIP Score calculation (heat map) (FIGS. 25A-25D). As shown in FIGS. 25A-25B, treatment with the biomass composition disclosed herein in combination with temozolomide induced synergistic antitumor activity. Notably, treatment with 1 (% v/v) of biomass composition alone (FIG. 25A, column 2, row 6) resulted in 9.1% cell survival, this was further reduced to 5% when 5 μM temozolomide was added. The inverse was also observed as 10 μM of temozolomide alone (FIG. 25A, column 5, row 4) resulted in 85.7% cell survival, which was further reduced to 67.3% when 0.5% v/v biomass composition was added. There was a ZIP synergy score of around +20, a mean of −1.04 and a p value of 2.07e-1. As shown in FIGS. 25C-25D, treatment with the biomass composition disclosed herein in combination with lomustine induced synergistic antitumor activity. Notably, treatment with 1 (% v/v) of biomass composition alone (FIG. 25C, column 2, row 6) resulted in 14.9% cell survival, this was further reduced to 8.2% when 5 μM lomustine was added. The inverse was also observed as 10 μM of lomustine alone (FIG. 25C, column 5, row 2) resulted in 86.4% cell survival, which was further reduced to 36.1% when 0.75% v/v biomass composition was added. There was a ZIP synergy score of around +20, a mean of −0.72 and a p value of 1.65e-1.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treatment or reduction of a brain cancer in a subject in need thereof, the method comprising administering to a subject a therapeutically effective dose of a biomass composition comprising a biomass material, wherein the biomass material comprises extract derived from:
at least two species of sea cucumber,
a species of seagrass,
a species of sea squirt, and
a species of sea urchin, and
wherein at least 80% w/w of the biomass material is from the at least two species of sea cucumber.

2. The method of claim 1, wherein the at least two species of sea cucumber comprise Holothuria scabra or Holothuria nobilis.

3. The method of claim 2, wherein the at least two species of sea cucumber comprise Holothuria scabra and Holothuria nobilis.

4. The method of claim 1, wherein the species of sea urchin comprises Heliocidaris erythrogramma.

5. The method of claim 1, wherein the species of sea squirt comprises Styela clava.

6. The method of claim 1, where the species of seagrass comprises Sargassum pallidum.

7. The method of claim 1, wherein the biomass material comprises extract derived from Holothuria scabra, Holothuria nobilis, Heliocidaris erythrogramma, Styela clava, and Sargassum pallidum to the subject.

8. The method of claim 1, wherein the brain cancer is a glioma.

9. The method of claim 8, wherein the glioma comprises a glioblastoma, an astrocytoma, or a diffuse intrinsic pontine glioma.

10. The method of claim 9, wherein the glioblastoma comprises a glioblastoma multiforme (GBM), a newly diagnosed glioblastoma, a giant cell glioblastoma, a gliosarcoma, a drug-resistant glioblastoma, a recurrent glioblastoma, a relapsed glioblastoma, or a refractory glioblastoma.

11. The method of claim 9, wherein the astrocytoma comprises a grade I astrocytoma, a grade II astrocytoma, a grade III astrocytoma, or a grade IV astrocytoma.

12. The method of claim 11, wherein the astrocytoma is a grade IV astrocytoma.

13. The method of claim 12, wherein the grade IV astrocytoma does not comprise an isocitrate dehydrogenase 1 (IDH1) mutation.

14. The method of claim 12, wherein the grade IV astrocytoma comprises an isocitrate dehydrogenase 1 (IDH1) mutation.

15. The method of claim 1, wherein the brain cancer is an oligodendroglioma, an ependymoma, a medulloblastoma, a choroid plexus carcinoma, a pineoblastoma, a meningioma, an acoustic neuroma, or a craniopharyngioma.

16. The method of claim 1, wherein the biomass composition is lyophilized.

17. The method of claim 1, wherein the biomass composition is a powder.

18. The method of claim 1, wherein the biomass composition is administered orally.

19. The method of claim 1, wherein the administering is daily or twice daily.

20. The method of claim 1, further comprising administering to the subject an additional cancer therapy.

21. The method of claim 20, wherein the additional cancer therapy is administered prior to, simultaneously with, or after the administering of the biomass composition.

22. The method of claim 21, wherein the additional cancer therapy comprises a chemotherapy or a radiation therapy.

23. The method of claim 22, wherein the additional cancer therapy comprises a chemotherapy and a radiation therapy.

24. The method of claim 22, wherein the chemotherapy comprises temozolomide, carmustine, bevacizumab, or lomustine.

25. The method of claim 22, wherein the radiation therapy comprises tumor treating fields (TTF), Intensity-modulated radiation therapy (IMRT), or Image-guided radiation therapy (IGRT).

26. The method of claim 1, wherein:
the at least two species of sea cucumber comprise Holothuria scabra and Holothuria nobilis, wherein from about 20% w/w to about 40% w/w of the biomass material comprises extract derived from the Holothuria scabra, wherein from about 25% w/w to about 50% w/w of the biomass material comprises extract derived from the Holothuria nobilis;
the species of seagrass comprises Sargassum pallidum, wherein from about 1% w/w to about 10% w/w of the biomass material comprises extract derived from the Sargassum pallidum;
the species of sea squirt comprises Styela clava, wherein from about 1% w/w to about 10% w/w of the biomass material comprises extract derived from the Styela clava; and
the species of sea urchin comprises Heliocidaris erythrogramma, wherein from about 1% w/w to about 10% w/w of the biomass material comprises extract derived from the Heliocidaris erythrogramma.

27. The method of claim 26, wherein:
40% w/w of the biomass material comprises extract derived from the Holothuria scabra,
45% w/w of the biomass material comprises extract derived from the Holothuria nobilis;
5% w/w of the biomass material comprises extract derived from the Sargassum pallidum;
5% w/w of the biomass material comprises extract derived from the Styela clava; and
5% w/w of the biomass material comprises extract derived from the Heliocidaris erythrogramma.

28. The method of claim 1, wherein the extract derived from the at least two species of sea cucumber, the species of seagrass, the species of sea squirt, and the species of sea urchin comprises a whole-body extract.

29. The method of claim 1, wherein the biomass composition further comprises a solubilizing agent.

30. The method of claim 29, wherein the solubilizing agent is dimethyl sulfoxide (DMSO).
